(19) United States
(12) Patent Application Publication
(10) Pub. No.: US 2023/0398155 A1
(43) Pub. Date: Dec. 14, 2023

(54) Human Adipose-Derived Stem Cell Exosomes And Their Packaging For Use At Room Temperature

(71) Applicant: The United States Government As Represented By The Department Of Veterans Affairs, Washington, DC (US)

(72) Inventor: NIKETA A. PATEL, Land O Lakes, FL (US)

(21) Appl. No.: 18/301,093

(22) Filed: Apr. 14, 2023

Related U.S. Application Data
(60) Provisional application No. 63/331,582, filed on Apr. 15, 2022.
(60) Provisional application No. 63/388,119, filed on Jul. 11, 2022.

(51) Int. Cl. A61K 35/28; A61P 17/02

(52) U.S. Cl. CPC A61K 35/28; A61P 17/02

(57) ABSTRACT

Disclosed are compositions comprising adipose stem cell (ASC) derived exosomes. Disclosed are lyophilized compositions comprising adipose stem cell (ASC) derived exosomes. Disclosed are methods of treating a wound comprising administering a composition comprising one or more adipose stem cell derived exosomes to a wound of a subject. Disclosed are methods of promoting wound healing comprising administering a composition comprising one or more adipose stem cell derived exosomes to a wound of a subject. Disclosed are methods of accelerating wound closure time comprising administering a composition comprising one or more adipose stem cell derived exosomes to a wound of a subject. Disclosed are methods of reducing oxidative stress in a human dermal fibroblasts (HDF) comprising contacting an HDF with a composition comprising one or more adipose stem cell derived exosomes. Disclosed are methods of increasing cell viability in a subject comprising administering a composition comprising one or more adipose stem cell derived exosomes to the subject.

### BACKGROUND

Battlefield injuries are 68% more likely to be a penetrating injury compared to civilian injuries (Blackburn 2009). The physical injuries may be sustained throughout the body and at times penetrate the head during combat. The long-term outcome of a traumatic, penetrating injury is manifold and ranges from physical wound healing or cognitive decline if the injury affects the brain. While multiple advances in wound dressing materials, surgical procedures and imaging promote healing, a noteworthy gap remains in the interventions available immediately in the narrow window of time following the injury which considerably improve the outcome of injury. There is an immediate need to develop agents that significantly promote wound healing and support positive long-term outcomes following injury which restore quality of life and combat readiness.

### BRIEF SUMMARY

Disclosed are compositions comprising adipose stem cell (ASC) derived exosomes. In some aspects the compositions further comprise a pharmaceutically acceptable carrier.

Disclosed are lyophilized compositions comprising adipose stem cell (ASC) derived exosomes.

Disclosed are methods of treating a wound comprising administering a composition comprising one or more adipose stem cell derived exosomes to a wound of a subject.

Disclosed are methods of promoting wound healing comprising administering a composition comprising one or more adipose stem cell derived exosomes to a wound of a subject.

Disclosed are methods of accelerating wound closure time comprising administering a composition comprising one or more adipose stem cell derived exosomes to a wound of a subject.

Disclosed are methods of reducing oxidative stress in a human dermal fibroblasts (HDF) comprising contacting an HDF with a composition comprising one or more adipose stem cell derived exosomes.

Disclosed are methods of increasing cell viability in a subject comprising administering a composition comprising one or more adipose stem cell derived exosomes to the subject.

Additional advantages of the disclosed method and compositions will be set forth, in part, in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A and 1B shows human adipose stem cells (hASC) from lean donor from either omental depot (OL) or subcutaneous depot (SL) were cultured. (A) Conditioned media was collected and exosomes (OLexo and SLexo) were isolated by precipitation (ultracentrifugation) followed by size exclusion columns. The lncRNA content was determined by real time qPCR using lncRNA Profiler. Amongst the consistently detected lncRNAs in exosomes, the lncRNAs MALAT1 and GAS5 were significantly higher (***p<0.001) in OLexo compared to SLexo. Relative quantification was calculated with SLexo as reference. (B) To obtain absolute quantities of lncRNAs GAS5 and MALAT1 contained per 1 μg of exosomes, droplet digital PCR (ddPCR) was performed. Results indicate significantly higher copy number of GAS5 and MALAT1 packaged within OLexo. Repeated 5 times.

FIG. 2 shows human dermal fibroblasts (HDF) plated at 90% confluency with Idibi cell inserts. These produce uniform scratches (gaps) to study wound healing. After 24 h, the cell insert was lifted and 2 μg of either OLexo or SLexo was added. Images were captured every 24 h upto 48 h for wound healing using the Keyence BX800 microscope. The images were uploaded and analyzed using Idibi analysis software. Experiment repeated 5 times with similar results.

FIG. 3 is an example of a container comprising a buffer and lyophilized human adipose stem cell derived exosomes.

FIG. 4 shows IVIS imaging of a F344 rat treated i.v. with 50 μg hASCexo-DiR.

FIG. 5 is an example of RNA FISH using ACD RNAscope probes in human dermal fibroblasts (HDF) treated with 2 μg hASCexo for 24 h. MALAT1 is present in nucleus while GAS5 is in cytoplasm. Repeated 5 times. The lines are drawn to where the MALAT1 and GAS5 expression merge which happens to be at the nucleus (identified by DAPI staining).

FIGS. 6A-6F show exosomes role in wound healing. A) exosomes isolated from hASCs were verified by western blot for hASC exosomal tetraspanin markers using antibodies against CD9, CD63, and CD81. The bands are representative of results obtained from experiments repeated five times. The graph represents ±SEM densitometric units. (B) The size and purity od hASC exosomes were evaluated using NanoSight v3.2.01 and (C) levels of long noncoding RNAs GAS5 and MALAT1 by absolute quantification by qPCR per 1 μg of exosomes across batches. (D) 1 μg of mCherry or GFP overexpression plasmids were transfected into hASCs from subcutaneous depot and exosomes (SLexo) were isolated from conditioned media. HDF cells were treated with 1 μg of exosomes and imaged using the Keyence microscope after 24 hours showing uptake of hASC exosomes carrying mCherry or GFP (scale bar 20 μm, n=3). (E) HDF cells were grown in a 35 mm plate with Ibidi μ-inserts to generate consistent gaps. Inserts were removed and HDF cells were treated with exosomes (Exo) or exosomes depleted of GAS5 (Exo-G5) or depleted of MALAT1 (Exo-M1). Gap was imaged at time 0 and re-imaged after 18 hours. Wound gap was measured using Image J and area was calculated in μm2 (n=3). Statistical analysis was performed by one-way ANOVA and significant p-values (<0.05) are indicated on graph. (F) 1 μg of GFP overexpression plasmid was transfected into hASCs from omental depot and exosomes (OLexo) were isolated from conditioned media. HDF cells were treated with 1 μg of exosomes and imaged using the Keyence microscope after 24 hours showing uptake of OLexo (scale n=3).

FIG. 7 shows HDF cells were seeded in a Seahorse XFp miniplate and treated with 100 μM H2O2 for 1 h, followed by medium change and treatment with hASC exosomes (Exo) or exosomes depleted of GAS5 (Exo-G5) for 18 h. A Mito Stress Test Assay was performed according to the manufacturer's instructions and repeated three times. The readings were normalized to the protein content of each well. Seahorse Wave software was used for analysis of oxygen consumption rate (OCR), basal respiration, percent spare respiratory capacity and coupling efficiency (n=3). Statistical analysis was performed by one-way ANOVA and significant p-values (<0.05) are indicated on the graph.

FIGS. 8A-8D show HDF cells were treated with 5 ng/mL LPS for 6 h and the medium was changed, followed by treatment with 1 μg hASC exosomes (Exo) or exosomes depleted of GAS5 (Exo-G5) for 18 h. (a) Acridine orange (AO) and propidium iodide (PI) dual staining was used to determine the viability of HDF cells (n=3). Statistical analysis was performed by one-way ANOVA and significant p-values (<0.05) are indicated on the graphs. (b) Immunocytochemistry was performed using Ki67 staining as a marker for cellular proliferation in HDF cells. Cells were also stained with nuclear marker DAPI and imaged with a Keyence BZx-810 microscope (scale bar=200 μm). Colocalization of Ki67 was determined using Keyence software (n=3). Statistical analysis was performed by one-way ANOVA and significant p-values (<0.05) are indicated on the graph. (c) HDF cells were grown in a 35 mm plate with Ibidi μ-inserts to generate consistent gaps. Inserts were removed and HDF cells were treated with 5 ng/mL LPS for 6 h followed by treatment with Exo or Exo-G5 for 18 h. Gaps were imaged at time 0 and reimaged after 18 h and 24 h. Wound gap was measured using Image J and area was calculated in μm2 (n=3). Statistical analysis was performed by one-way ANOVA and significant p-values (<0.05) are indicated on the graph. (d) RNA was isolated from HDF cells and SYBR Green real-time qPCR was performed using primers for IL1β and IL6; relative quantification was calculated, normalizing to GAPDH (n=3). Statistical analysis was performed by one-way ANOVA and significant p-values (<0.05) are indicated on the graph.

FIGS. 9A-9E show HDF cells were treated with 5 ng/mL LPS for 6 h followed by hASC exosomes or exosomes depleted of GAS5 for 18 h. LPS was maintained in the media and cells were harvested after 4 days. RNA was isolated and PCR was run using the Human Inflammation Array (Qiagen, cat #PAHS-077Z). (A) Using GeneGlobe online data analysis software (Qiagen), a heatmap was generated from array data showing differentially expressed genes. (B) Genes identified from the heatmap showing patterns correlating to LPS, Exo, and Exo-G5 treatments were verified further by real-time qPCR. Relative quantification (RQ) was determined using a control sample as reference (n=3). Statistical analysis was performed by one-way ANOVA and significant p-values (<0.05) are indicated on the graph. (C) ingenuity pathway analysis was performed on array data identifying canonical pathways influenced by GAS5 presence in exosomes. Changes in (D) the TLR pathway and (E) wound healing pathway were significantly altered in response to exosome and GAS5-depleted exosome treatment

FIGS. 10A and 10B show (A) HDF cells were treated with 5 ng/mL LPS for 4 days. SYBR Green real-time qPCR was performed using GAS5 primers. Relative quantification (RQ) was determined using a control sample as reference (n=3). Statistical analysis was performed by t-test and significant p-values (<0.05) are indicated on the graph. (B) GAS5 was depleted in HDF cells by transfecting 25 nM GAS5 siRNA or negative control siRNA (siRNA Con) for 48 h. RNA was isolated and real-time qPCR was performed using primers specific for GAS5, TLR7, IFNα, IL1β, or TNFα. Relative quantification (RQ) was determined using a control sample as reference (n=3). Statistical analysis was performed by t-test and significant p-values (<0.05) are indicated on the graph.

FIG. 11 shows HDF cells were grown on a collagen scaffold to mimic 3D wound healing. Chronic inflammation was induced for 4 days using 5 ng/mL LPS for 6 h followed by treatment with hASC exosomes (Exo) or exosomes depleted of GAS5 (Exo-G5) for 18 h. LPS was maintained in the media along with the treatment. The 3D wound model was maintained at 37° C. and wound closure was imaged every 24 h using a Keyence BX810 microscope (n=3). Wound closure was calculated each day using a Keyence's Cell migration assay with Hybrid cell count software to calculate wound gap area. Statistical analysis was performed by one-way ANOVA and significant p-values (<0.05) are indicated on the graph.

FIGS. 12A and 12B show in vivo wound healing model using SL and OL exosomes. (A) Digital photographs were taken on days 0 and 7 and wound area measured using ImageJ as well as using calipers and tracing. At the designated days of the experimental design, wound measurements were taken by digital photos and processed using ImageJ software. (B) To evaluate the outcome of hASCexo treatment in vivo, Fisher F344 rats underwent three 6-mm excisional wounds. Young adult (6-month-old) rats were divided into three groups (n=6; 3M and 3F) and treated with hASCexo: (1) Control (PBS vehicle treatment) (2) SL exosomes treatment (3) OL exosomes treatment. 100 μg of hASCexo were applied topically to each wound on alternate days along with dressing changes beginning on the day of surgery. Quantification of wounds over the time course of healing is done by calculating the circumference mm2 at day (using the measurement tool of software) using a formula: punch biopsy area=(π) r2. Data is graphed with data presented as percentage of initial wound area vs wound surface area on a particular day (percent wound closure). Statistical analysis was performed by one-way ANOVA and significant p-values (***<0.001) are indicated on graph. Analysis revealed a statistically significant difference between control and exosomes treated wounds.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an adipose stem cell derived exosome” includes a plurality of such adipose stem cell derived exosomes, reference to “the composition” is a reference to one or more compositions and equivalents thereof known to those skilled in the art, and so forth.

As used herein, the term “subject” or “patient” can be used interchangeably and refer to any organism to which a composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

By “treat” is meant to administer a composition of the invention to a subject, such as a human or other mammal (for example, an animal model), that has a wound, such as a chronic wound.

As used herein, the terms “administering” and “administration” refer to any method of providing a disclosed composition to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed protein so as to treat a subject or induce an immune response. In an aspect, the skilled person can also alter or modify an aspect of an administering step so as to improve efficacy of a disclosed composition.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods

Disclosed are methods of using one or more of the disclosed compositions. In some aspects, the composition further comprises a pharmaceutically acceptable carrier. Thus, the compositions include pharmaceutical compositions.

Disclosed are methods of using a composition comprising one or more (e.g. at least one) adipose stem cell derived exosome. In some aspects, the one or more adipose stem cell derived exosome is a human adipose stem cell (hASC) exosomes.

In some aspects, the one or more adipose stem cell derived exosome comprises one or more long noncoding RNAs (lncRNA). In some aspects, the lncRNA can be growth-arrest specific-5, GAS5 and/or metastasis-associated lung adenocarcinoma transcript 1 (MALAT1). One or both of GAS5 and MALAT1 can be present in the adipose stem cell derived exosome.

In some aspects, the adipose stem cell derived exosome is cell free.

In some aspects, IL1β and IL6 levels were decreased in a subject compared to before administering one of the disclosed compositions. In some aspects, TLR7, CCL17 and ITGB2 levels were decreased compared to before administering the composition.

In some aspects, the one or more adipose stem cell derived exosomes express CD9, CD63, and CD81. Thus, antibodies that bind to these markers can be used to identify exosomes.

In some aspects, the one or more adipose stem cell derived exosomes comprise a targeting moiety. In some aspects, the targeting moiety can be expressed on the surface of the one or more adipose stem cell derived exosomes. In some aspects, the targeting moiety comprises a peptide which binds to a moiety (e.g. ligand) present on a cell to be targeted.

1. Methods of Promoting Wound Healing

Disclosed are methods of promoting wound healing comprising administering a composition comprising one or more adipose stem cell derived exosomes to a wound of a subject.

In some aspects, the wound is a dermal wound. In some aspects, the wound is a cut, laceration or incision. In some aspects, the wound is a chronic recalcitrant wound.

In some aspects, the subject has an underlying dermal inflammation. In some aspects, the underlying dermal inflammation results in impaired natural wound healing.

In some aspects, dermal fibroblasts in the wound take up the one or more adipose stem cell derived exosomes. In some aspects, the adipose stem cell derived exosomes are distributed within the cytoplasm and nucleus of the dermal fibroblasts.

In some aspects, the wound has an accelerated wound closure. In some aspects, accelerated wound closure can mean a faster wound closure compared to a wound that does not receive the adipose stem cell derived exosomes. In some aspects, accelerated wound closure can mean a faster wound closure compared to a wound that receives any other treatment other than the adipose stem cell derived exosomes. In some aspects, accelerated wound closure can mean a faster wound closure compared to a wound that receives no treatment. In some aspects, a faster wound closure can mean the wound closes at least 1, 2, 3, 4, 5, 6, 7, 8, 9, 10, 11, 12, 13, 14, 15, 16, 17, 18, 19, 20, 21, 22, 23, 24 hours before an untreated or non-adipose stem cell derived exosome treated wound. In some aspects, a faster wound closure can mean the wound closes at least 1, 2, 3, 4, 5, 6, or 7 days before an untreated or non-adipose stem cell derived exosome treated wound. In some aspects, a faster wound closure can mean the wound closes at least 1, 2, 3, or 4 weeks before an untreated or non-adipose stem cell derived exosome treated wound.

In some aspects, the administration is a topical administration.

2. Methods of Treating a Wound

Disclosed are methods of treating a wound comprising administering a composition comprising one or more adipose stem cell derived exosomes to a wound of a subject. In some aspects, the composition comprising one or more adipose stem cell derived exosomes can be any of the compositions described herein.

In some aspects, the wound is a dermal wound. In some aspects, the wound is a cut, laceration or incision. In some aspects, the wound is a chronic recalcitrant wound.

In some aspects, the subject has an underlying dermal inflammation. In some aspects, the underlying dermal inflammation results in impaired natural wound healing.

In some aspects, dermal fibroblasts in the wound take up the one or more adipose stem cell derived exosomes. In some aspects, the adipose stem cell derived exosomes are distributed within the cytoplasm and nucleus of the dermal fibroblasts.

In some aspects, the wound has an accelerated wound closure. In some aspects, accelerated wound closure can mean a faster wound closure compared to a wound that does not receive the adipose stem cell derived exosomes. In some aspects, accelerated wound closure can mean a faster wound closure compared to a wound that receives any other treatment other than the adipose stem cell derived exosomes. In some aspects, accelerated wound closure can mean a faster wound closure compared to a wound that receives no treatment. In some aspects, a faster wound closure can mean the wound closes at least 1, 2, 3, 4, 5, 6, 7, 8, 9, 10, 11, 12, 13, 14, 15, 16, 17, 18, 19, 20, 21, 22, 23, 24 hours before an untreated or non-adipose stem cell derived exosome treated wound. In some aspects, a faster wound closure can mean the wound closes at least 1, 2, 3, 4, 5, 6, or 7 days before an untreated or non-adipose stem cell derived exosome treated wound. In some aspects, a faster wound closure can mean the wound closes at least 1, 2, 3, or 4 weeks before an untreated or non-adipose stem cell derived exosome treated wound.

In some aspects, the administration is a topical administration.

In some aspects, the disclosed methods of treating a wound comprise complete wound closure or partial wound closure. For example, any improvement of the wound in response to a composition comprising one or more adipose stem cell derived exosomes is considered treating.

3. Methods of Accelerating Wound Closure Time

Disclosed are methods of accelerating wound closure time comprising administering a composition comprising one or more adipose stem cell derived exosomes to a wound of a subject. In some aspects, the composition comprising one or more adipose stem cell derived exosomes can be any of the compositions described herein.

In some aspects, the wound is a dermal wound. In some aspects, the wound is a cut, laceration or incision. In some aspects, the wound is a chronic recalcitrant wound.

In some aspects, the subject has an underlying dermal inflammation. In some aspects, the underlying dermal inflammation results in impaired natural wound healing.

In some aspects, dermal fibroblasts in the wound take up the one or more adipose stem cell derived exosomes. In some aspects, the adipose stem cell derived exosomes are distributed within the cytoplasm and nucleus of the dermal fibroblasts.

In some aspects, the wound has an accelerated wound closure. In some aspects, accelerated wound closure can mean a faster wound closure compared to a wound that does not receive the adipose stem cell derived exosomes. In some aspects, accelerated wound closure can mean a faster wound closure compared to a wound that receives any other treatment other than the adipose stem cell derived exosomes. In some aspects, accelerated wound closure can mean a faster wound closure compared to a wound that receives no treatment. In some aspects, a faster wound closure can mean the wound closes at least 1, 2, 3, 4, 5, 6, 7, 8, 9, 10, 11, 12, 13, 14, 15, 16, 17, 18, 19, 20, 21, 22, 23, 24 hours before an untreated or non-adipose stem cell derived exosome treated wound. In some aspects, a faster wound closure can mean the wound closes at least 1, 2, 3, 4, 5, 6, or 7 days before an untreated or non-adipose stem cell derived exosome treated wound. In some aspects, a faster wound closure can mean the wound closes at least 1, 2, 3, or 4 weeks before an untreated or non-adipose stem cell derived exosome treated wound. In some aspects, omental-derived adipose stem cell derived exosomes promote healing at a significantly faster rate than subcutaneous-derived adipose stem cell derived exosomes.

In some aspects, the administration is a topical administration.

4. Methods of Reducing Oxidative Stress in a Human Dermal Fibroblasts

Disclosed are methods of reducing oxidative stress in a human dermal fibroblasts (HDF) comprising contacting an HDF with a composition comprising one or more adipose stem cell derived exosomes. In some aspects, the composition comprising one or more adipose stem cell derived exosomes can be any of the compositions described herein.

In some aspects, dermal fibroblasts in the wound take up the one or more adipose stem cell derived exosomes. In some aspects, the adipose stem cell derived exosomes are distributed within the cytoplasm and nucleus of the dermal fibroblasts.

In some aspects, the methods can be in vivo or in vitro. Thus, in some aspects, the contacting an HDF with a composition comprising one or more adipose stem cell derived exosomes can include administering a composition comprising one or more adipose stem cell derived exosomes to a subject, wherein the subject comprises HDF. In some aspects, the contacting an HDF with a composition comprising one or more adipose stem cell derived exosomes can include contacting the HDF with the composition in culture.

5. Methods of Increasing Cell Viability

Disclosed are methods of increasing cell viability in a subject comprising administering a composition comprising one or more adipose stem cell derived exosomes to the subject. In some aspects, the subject has inflammation, for example, from a wound. In some aspects, without treatment, a wound can result in decreased cell viability (likely due to inflammation). Thus, in some aspects, treating with a composition comprising one or more adipose stem cell derived exosomes can increase or improve cell viability. In particular, in some aspects, the presence of GAS5 in the exosomes increases cell viability.

### C. Compositions

Disclosed are compositions comprising adipose stem cell (ASC) derived exosomes. Exosomes derived from different adipose depots have been characterized in Patel et al. Stem Cell Investigation, 2016 Jan. 31; 3:2, hereby incorporated by reference in its entirety.

Adipose stem cells, or adipose-derived stem cells (ASCs), are an important stem cell type separated from adipose tissue, with the properties of multi-lineage differentiation, easy availability, high proliferation potential, and self-renewal. Exosomes are involved in intercellular communication regulating the biological behaviors of cells, such as angiogenesis, immune modulation, proliferation, and migration. ASC-derived exosomes (ASC-exos) are important components released by ASCs paracrine, possessing multiple biological activities.

In some aspects, the adipose stem cell derived exosomes are subcutaneous-derived adipose stem cell derived exosomes. In some aspects, the adipose stem cell derived exosomes are omental-derived adipose stem cell derived exosomes. In some aspects, the adipose stem cell derived exosomes can be a combination of subcutaneous-derived adipose stem cell derived exosomes and omental-derived adipose stem cell derived exosomes.

In some aspects, the adipose stem cell derived exosome is a human adipose stem cell derived exosome (hASC).

In some aspects, the adipose stem cell derived exosome is cell free.

In some aspects, the adipose stem cell derived exosome comprises a targeting moiety. In some aspects, the targeting moiety comprises a peptide which binds to a moiety present on a cell to be targeted. For example, a targeting moiety can be an antibody that bind to its ligand present on the cell to be targeted. In some aspects, the adipose stem cell derived exosome comprises a labeling moiety. In some aspects, the labeling moiety can be a fluorescent marker.

Disclosed are subcutaneous-derived adipose stem cell derived exosomes comprising lncRNA growth-arrest specific-5 (GAS5), wherein GAS5 is overexpressed. Disclosed are subcutaneous-derived adipose stem cell derived exosomes comprising lncRNA metastasis-associated lung adenocarcinoma transcript 1 (MALAT1), wherein MALAT1 is overexpressed. In some aspects, the subcutaneous-derived adipose stem cell derived exosomes comprise both GAS5 and MALAT1, wherein GAS5 and MALAT1 are both overexpressed. In some aspects, overexpressed can mean expression levels are higher than what is found in naturally occurring subcutaneous-derived adipose stem cell derived exosomes. In some aspects, subcutaneous-derived adipose stem cell derived exosomes per 1 μg contain approximately 0.46 copies of GAS5 and 0.43 copies of MALAT1. In some aspects, omental-derived adipose stem cell derived exosomes per 1 μg contain approximately copies of GAS5 and 1.45 copies of MALAT1. In some aspects, overexpression in subcutaneous-derived adipose stem cell derived exosomes can be at least 3-4 fold of GAS5 and/or MALAT1 per 1 μg of exosome.

In some aspects, subcutaneous-derived adipose stem cell derived exosomes can be altered to more closely resemble omental-derived adipose stem cell derived exosomes. In some aspects, altering subcutaneous-derived adipose stem cell derived exosomes can comprise increasing the levels of GAS5 and/or MALAT1.

1. Pharmaceutical Compositions

Disclosed are pharmaceutical compositions comprising any one of the compositions disclosed herein and a pharmaceutically acceptable carrier.

Disclosed are pharmaceutical compositions comprising at least one adipose stem cell derived exosome and a pharmaceutically acceptable carrier.

In some aspects, the at least one adipose stem cell derived exosome can be any of those described herein.

As disclosed herein, are pharmaceutical compositions, comprising the compositions disclosed herein. In an aspect, the pharmaceutical composition can comprise adipose stem cell derived exosomes. In some aspects, the pharmaceutical compositions can further comprise a pharmaceutically acceptable carrier. In some aspects, the pharmaceutical compositions described herein can be sterile and contain any of the disclosed compositions for producing the desired response in a unit of weight or volume suitable for administration to a subject. In some aspects, the pharmaceutical compositions can contain suitable buffering agents, including, e.g., acetic acid in a salt; citric acid in a salt; boric acid in a salt; and phosphoric acid in a salt.

When administered, the composition can be administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term denotes an organic or inorganic ingredient, natural or synthetic, with which the active ingredient is combined to facilitate the application. The components of the pharmaceutical compositions also are capable of being co-mingled with the disclosed compositions, and with each other, in a manner such that there is no interaction which would substantially impair the desired pharmaceutical efficacy.

As used herein, the term “pharmaceutically acceptable carrier” refers to solvents, dispersion media, coatings, antibacterial, isotonic and absorption delaying agents, buffers, excipients, binders, lubricants, gels, surfactants that can be used as media for a pharmaceutically acceptable substance. The pharmaceutically acceptable carriers can be lipid-based or a polymer-based colloid. Examples of colloids include liposomes, hydrogels, microparticles, nanoparticles and micelles. The compositions can be formulated for administration by any of a variety of routes of administration, and can include one or more physiologically acceptable excipients, which can vary depending on the route of administration. Any of the compositions described herein can be administered in the form of a pharmaceutical composition.

As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. The compositions can also include additional agents (e.g., preservatives).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions and sterile packaged powders. The active ingredient can be any of the growth hormone releasing hormone peptides described herein in combination with one or more pharmaceutically acceptable carriers. As used herein “pharmaceutically acceptable” means molecules and compositions that do not produce or lead to an untoward reaction (i.e., adverse, negative or allergic reaction) when administered to a subject as intended (i.e., as appropriate).

In some aspects, administration of compositions disclosed herein can be administered to mammals other than humans, e.g., for testing purposes or veterinary therapeutic purposes, can be carried out under substantially the same conditions as described above.

2. Lyophilized Compositions

In some aspects, the disclosed compositions can be lyophilized.

Disclosed are lyophilized compositions comprising an adipose stem cell derived exosome. In some aspects, adipose stem cell derived exosome can be any of the adipose stem cell derived exosomes described herein. For example, in some aspects the human adipose stem cell derived exosome can be omental-derived. In some aspects the human adipose stem cell derived exosome can be subcutaneous-derived.

In some aspects, the disclosed lyophilized compositions comprise less than 20%, 15%, 10%, or 5% residual water. In some aspects, the disclosed lyophilized compositions comprise ≤5% residual water.

In some aspects, the disclosed lyophilized compositions comprise trehalose. In some aspects, the trehalose is present at a concentration of less than 2M, 1.5M, 1M, 0.5M or

Disclosed are lyophilized compositions can be stored in the containers disclosed herein. In some aspects, the lyophilized compositions can be sealed inside a sterile package.

In some aspects, the lyophilized compositions disclosed herein can be stable for at least three weeks at room temperature. In some aspects, the lyophilized compositions can be stable for at least three months at room temperature. In some aspects, the lyophilized compositions can be stable for 1, 2, 3, 4, 5, 6, 7, 8, 9, 10, 11, 12, 24, 36, 48, or 60 months at room temperature.

In some aspects, the lyophilized compositions disclosed herein can be reconstituted resulting in a reconstituted tissue. The described lyophilized compositions can be reconstituted using standard techniques known in the art. In some aspects, reconstituting refers to rehydrating. Thus, the disclosed lyophilized compositions can be reconstituted or rehydrated using water, saline, a buffer such as, but not limited to phosphate buffered saline (PBS), in a solution comprising a stabilizing agent such as, but not limited to bovine serum albumin (BSA), Plasma-Lyte A or other clinically available electrolyte solutions, with human bodily fluids or a combination thereof. For example, lyophilized compositions can be applied directly to a wound or tissue injury on a subject and the subject's bodily fluids can reconstitute. In some aspects, a combination of bodily fluids and another known rehydrating solution can be used.

D. Containers

Disclosed are containers comprising any one of the compositions disclosed herein. In some aspects, the container is a vial. In some aspects, the disclosed containers comprise one or more of the disclosed lyophilized compositions and a buffer solution. In some aspects, the composition and/or buffer solution can be sterile.

Disclosed are vials comprising a vial body having a first body portion that defines a first interior volume; and a second body portion that defines a second interior volume; a buffer solution positioned within one of the first interior volume or the second interior volume; a composition comprising one or more of the compositions described herein, wherein the composition is positioned within the other of the first interior volume or the second interior volume; and a barrier between the first interior volume and the second interior volume, wherein the barrier prevents liquid communication between the first interior volume and the second interior volume, wherein the second body portion is selectively rotatable relative to the first body portion to displace or pierce the barrier and establish liquid communication between the first interior volume and the second interior volume. In some aspects, the composition present in the first interior volume or the second interior volume is any one of the lyophilized compositions disclosed herein.

In some aspects, the first body portion defines a bottom end of the vial body, wherein the second body portion defines a top end of the vial body, wherein the top end of the vial body comprises an opening in liquid communication with the second interior volume, and wherein the vial further comprises a tab that forms a liquid seal around the opening of the top end of the vial body, wherein the vial is selectively detachably coupled to the vial body to permit removal of the liquid seal around the opening of the top end of the vial body.

### E. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the compositions disclosed herein. In some aspects, the kits can comprise one or more of the containers disclosed herein. The kits also can contain instructions for how to use the containers.

### EXAMPLES

### A. Example 1: Human Adipose-Derived Stem Cell Exosomes' and their Packaging for Use at Room Temperature

Human adipose-derived stem cell (hASC) exosomes can be used to treat injuries. It accelerates the wound healing 50% faster with decreased scar tissue formation. This is of great benefit to soldiers on the battlefield or worldwide for injuries. Previous descriptions of wound healing by exosomes were made using the subcutaneous fat as a source of hASC. However, the limitation is in the quantities available.

However, since exosomes are biologicals they need to be stored at 4° C. for up to 4 weeks or at −20° C. for up to 6 months. To be able to carry it to the battlefront and apply immediately after injury, a better storage and transport packaging is needed.

This study explored the omental depot as a source for hASC and its exosomes which is available in large quantities as surgical waste in almost all surgical procedures. Further, the content of exosomes were evaluated as a measure of wound healing results.

1. Background

Stem cells in regenerative medicine: Adult mesenchymal stem cells derived from bone marrow or adipose tissue have shown potential in regenerative medicine. While both sources are comparable with the stem cell markers, deriving from bone marrow poses several issues such as painful procedure and limited number of stem cells. The advantages of human adipose-derived stem cells (hASC) over other stem cells lies in its availability (obtained from surgeries as discarded adipose tissue and from liposuction) and ease of isolation and maintenance. The hASC can differentiate into adipocytes, osteoblasts, muscle or neural cells. The isolation of hASC from adipose tissue, where hASC reside in the stromal vascular fraction, has been mastered. The stem cell antigens and markers have been characterized and its renewal capacity and differentiation potential evaluated. Initially hASC were used as intravenous transplant in an in vivo model of traumatic brain injury and was compare with treatment of hASC secretome. It was shown that this was a promising approach to treat TBI and importantly, it was shown that the hASC secretome promotes recovery post injury. This is highly significant as it mitigates the safety issues posed by stem cells such as migrating to different organs or undergo differentiation into a different phenotype.

Over the years, the secretome of hASC has been defined. It was shown that hASC secretome contains exosomes which are small extracellular vesicles (80-100 nm) that regulate fundamental cellular functions in recipient cells. hASC, its secretome and its exosomes have been characterized, and it has been demonstrated that exosomes secreted from hASC contained cargo driving its regenerative therapeutic ability.

Advantages of hASC exosomes is that it provides a cell-free regenerative approach over other therapies such as nanoparticles or stem cells. The natural biocomponents of exosomes are not toxic, no transfections or carriers are required and exosomes are efficiently taken up by the site of injury by known mechanisms and localize to the site of injury thus providing significant advantage as a therapeutic in treating injuries.

Exosomes are stable nanovesicles and can be stored at −20° C. for 6 months or 2 weeks at 4° C. Other groups have demonstrated that exosomes can be stored at room temperature and this significantly adds to the advantage of its use in the battlefront injuries. Charoenviriyakul et al (2018) have demonstrated that exosomes can be lyophilized in the presence of trehalose and stored at room temperature for 4 weeks. The lyophilized exosomes retain their protein and RNA cargo and mediate their effects when injected intravenously into mice

2. Significance

Battlefield injuries are 68% more likely to be a penetrating injury compared to civilian injuries (Blackburn 2009). The physical injuries may be sustained throughout the body and at times penetrate the head during combat. The long-term outcome of a traumatic, penetrating injury is manifold and ranges from physical wound healing or cognitive decline if the injury affects the brain. While multiple advances in wound dressing materials, surgical procedures and imaging promote healing, a noteworthy gap remains in the interventions available immediately in the narrow window of time following the injury which considerably improve the outcome of injury. There is an immediate need to develop agents that significantly promote wound healing and support positive long-term outcomes following injury which restore quality of life and combat readiness. Towards this goal, a therapeutic approach using human adipose stem cells derived exosomes (hASCexo) was evaluated. The data shows a profound hASCexo mediated alteration of genome wide expression of key pathways including pro-inflammatory, transcriptional activation of regenerative pathways, and reduction in cell death. The data shows that hASCexo enable wound healing and recovery of function by modulating genomic pathways following injury. There is in vivo data in models of ischemic wound healing and traumatic brain injury.

3. Results

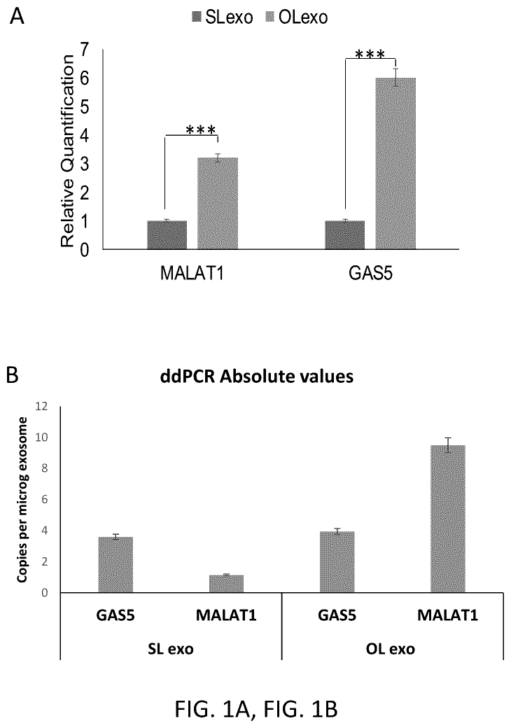

Described herein is an analysis of exosomes derived from subcutaneous vs omental fat depot of lean donors showed differences in their cargo. Notably, the long noncoding RNAs (lncRNAs—which are master regulators of gene expression and function) GAS5 and MALAT1 were packaged at a higher level in exosomes derived from omental hASC (OLexo) (FIG. 1).

The wound healing and regenerative potential of OLexo and SLexo were determined. Since GAS5 and MALAT1 were significantly increased in OLexo compared to SLexo, it was also determined whether depletion of either lncRNA MALAT1 or lncRNA GAS5 in OLexo impairs the wound healing ability of OLexo in these experiments. Human dermal fibroblasts (HDF) were plated at 90% confluency with Idibi cell inserts and wound healing assay was performed.

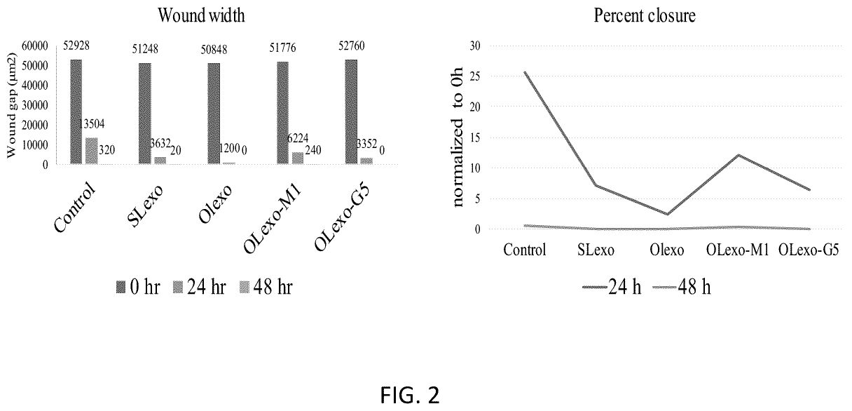

The data (FIG. 2) shows that OLexo increases wound healing at a significantly faster rate compared to exosomes derived from subcutaneous hASC (SLexo). Further, the results indicate that lncRNA MALAT1 and GAS5 contained in OLexo are drivers of the regenerative potential of OLexo.

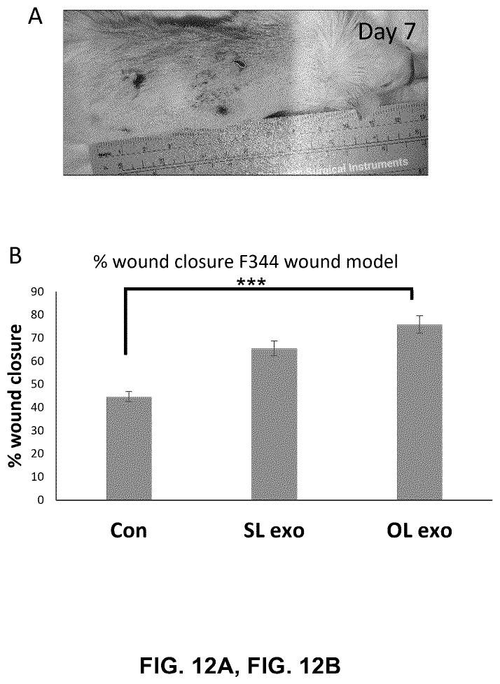

FIG. 12 shows that SLexo and OLexo significantly improves wound healing and percent wound closure in an in vivo model of excisional wounds. The data shows that OLexo improve wound closure faster compared to SL exo. The control PBS-treated wounds showed 44% closure on day 7, 65% with SL exosomes treatment and 76% with OL exosomes treatment. This demonstrates that SL and OL exosomes are safe, efficiently taken up, not toxic and promote accelerated healing of dermal wounds in vivo. Using F344 rats, full-thickness excisional wounds were created on the dorsum and the panniculus carnosus muscle was removed from the wound bed by dissecting just above the muscle fascia. The dorsal wounds were ischemic. Wound contraction was limited by anchoring a silicone ring and suturing. Wounds were then dressed in Tegaderm transparent film secured with skin adhesive after topical application of the exosomes. The rats were fitted with cones over their necks to prevent them from disturbing the wounds. At the designated days of the experimental design, wound measurements were taken by digital photos and processed using ImageJ software. Quantification of wounds over the time course of healing is done by calculating the circumference mm² at day (using the measurement tool of software) using a formula: punch biopsy area=(π) r². Data is graphed with data presented as percentage of initial wound area vs wound surface area on a particular day (percent wound closure).

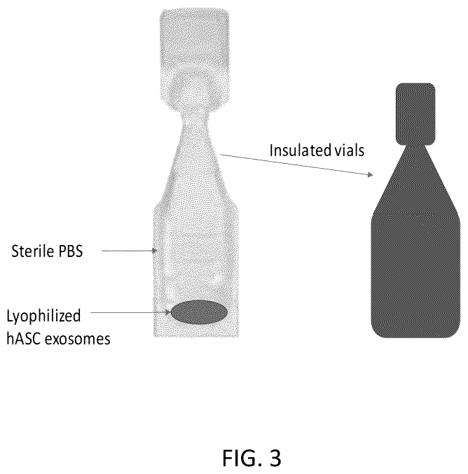

The OLexo exosomes can be lyophilized in the presence of trehalose and stored at room temperature for 4 weeks (Charoenviriyakul et al 2018). Packaging in single use vials is described herein. The lyophilized hASC exosomes are contained in bottom pouch with sterile PBS in upper pouch. Twisting the vial releases the PBS and thereby hydrates the exosomes. The top tab can then be snapped off and exosomes can be applied to the injury. The vials are made of insulation material to extend room temperature use. See FIG. 3 as an example of the packaging vials.

### B. Example 2: Labeling of Exosomes

1. Experiment 1

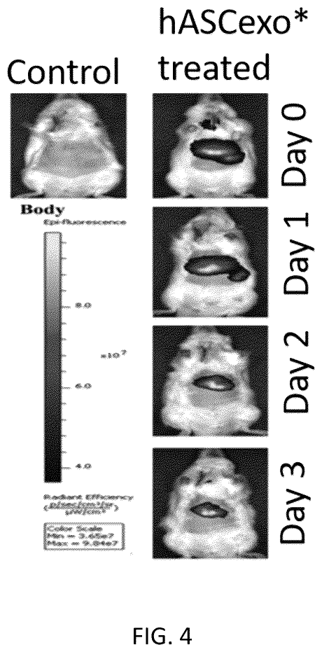

Wound healing promoted by hASCexo efficacy is dependent on the pharmacodynamics of hASCexo. The hASCexo can be labeled with DiR for in vivo labeling and visualization in the body using IVIS Illumina III (FIG. 4). Additionally, GFP is incorporated in hASCexo (overexpression hASC and secreted within its exosomes). Dual DiR and GFP labeled hASCexo (hASCexo*) can be used in the absorption, distribution, metabolism and excretion (ADME) studies as follows.

The wounds are treated topically in the flap model with F344 rats with the optimal concentration (determined in SA2a) of labeled hASCexo* on the day of wound. Twelve F344 Fisher rats (equal M/F) can be used per group. Animals can be imaged every 24 hours up to 3 days (total 3 timepoints). Cohorts are (1) Control (flap wound with PBS treatment) (2) hASCexo* treatment day1 (3) hASCexo* treatment day2 (4) hASCexo* treatment day3.

Exosome internalization is needed for the RNA cargo to be released in order to promote wound healing (cellular response) and it has been previously shown to be mediated by membrane fusion or endocytosis. Absorption and internalization of hASCexo* can be determined by IVIS imaging of DiR dye which can also be used to assess the in vivo distribution of hASCexo*. To evaluate the systemic absorption, the rats can be housed individually in metabolic cages (Phenomaster™) for 24 hours before each timepoint. Body metabolism is continuously monitored with O2 intake:CO2 exhaled to calculate respiratory quotient and metabolic rate, calorimetric parameters together with food and water intake. Body weight, temperature, locomotor activity, sleep/awake cycle of rats are monitored and analyzed throughout the experiment. Urine and feces are collected separately in these cages to determine systemic absorption and excretion of hASCexo*. The uptake locally and systemic is calculated to evaluate the absorption and distribution patterns. The outcomes can be compared with progress in wound healing.

However, since the hASCexo* are applied topically, the objective is to maximize its concentration at the site of the wound with minimal systemic uptake. Bioavailability (BA) for topical drugs is defined as the rate and extent to which the drug is absorbed from the topical formulation and becomes available at the site of action. This is calculated as amount of drug permeating per unit area (Q/A) versus time. Hence, the levels of hASCexo* are measured within the wound (as amount of hASCexo*/mm of wound) and are graphed versus time in days. To validate these findings, ex vivo skin explant studies can be performed. 0.4 mm thick skin tissue from the rats can be treated with optimized dose of hASCexo* and analyzed every 4 hours. BA can be calculated as above. The amount absorbed and extent of absorption can also be quantified using the Zeiss Light Sheet Z.1 microscope for whole organ imaging (VA core equipment, PI is Director of Core). This Multiview light sheet fluorescence microscope can image large living samples and produce 3D and 4D images. The optical sections are obtained at subcellular resolution in seconds. The software automatically calculates the Z-stacks and fluorescence measures.

Wound measurements using digital imaging can be done. Rats are euthanized and all organs as well as tissue around the wound is harvested. Wounds can be excised to include some surrounding healthy tissue. Half can be used for slicing sections and immunohistochemistry and other half can be immediately snap-frozen and stored at −80° C. for molecular measurements (details in SA2c). Controls can be administration of DiR alone and unlabeled exosomes. To determine the uptake, metabolism and turnover of exosomes, Real time qPCR for GFP and human GAS5 and MALAT1 (primers specifically determine levels of human origin i.e. from hASCexo compared to endogenous rat lncRNAs) can be performed.

Western blot analysis can be done to determine the levels of tetraspanins CD9, CD63 and CD81 (specific markers of hASCexo).

2. Experiment 2

Wound healing promoted by hASCexo is dependent on the lncRNAs GAS5 and MALAT1 contained as cargo of hASCexo. An important aspect of testing the hASCexo treatment as a strong therapeutic agent in wound healing and move it to closer to clinical trial is understanding the mode of action and the underlying molecular mechanism. lncRNAs GAS5 and MALAT1 contained in hASCexo are drivers of tissue repair and regeneration. By manipulating the levels of lncRNAs GAS5 (G5) and/or MALAT1 (M1) in hASCexo, the efficacy of hASCexo to promote wound closure and healing can be accelerated. An important undertaking in this project is to translate the therapy from bench-to-bedside. Hence, results of the in vitro defining the targets and manipulating the hASCexo lncRNA cargo can be incorporated and evaluated in this in vivo aim.

Eighteen F344 Fisher rats (equal M/F) can be used per group. This numbers takes into consideration the IHC and biochemical analysis proposed herein. The wounds are treated in the flap model in F344 rats (equal M/F) with the optimal concentration (as determined in SA2a) of hASCexo treatment. To validate that GAS5 and MALAT1 contained in hASCexo are the driving force of hASCexo mediated recovery, their levels within the exosomes can be manipulated (deplete (−) or over-express (oe)). Wounds can be treated with optimized dose of hASCexo-G5, hASCexo-M1, hASCexo_{-(M1 and G5)}, hASCexo_{G5oe}, hASCexo_{M1oe}, hASCexo_{(M1 and G5)oe}.

The seven hASCexo formulations can be applied topically on the day of injury and treatment can be repeated everyday with changes to the dressing. Wound measurements and laser doppler imaging can be taken every 3 days until day 21. Rats can be euthanized for tissue collection on days 7, 14 and 21. Wounds can be excised to include some surrounding healthy tissue. ⅓^{rd} can be used for immunohistochemistry, ⅓^{rd} to determine oxidative stress and ⅓^{rd} snap-frozen and stored at −80° C. for biochemical measurements.

To evaluate wound healing, digital images and software to calculate percent of area healed can be used. Absolute quantification of amounts of GAS5 and MALAT1 taken up by wounds can be analyzed with rate of wound closure.

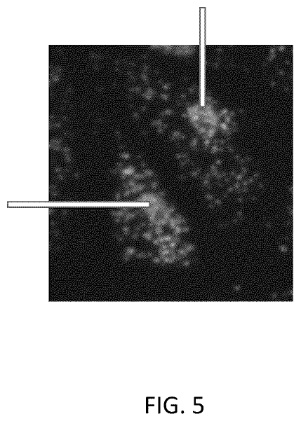

To evaluate uptake of exosomes, sections can be stained with hASC exosome markers CD9, CD63 and CD81 or used in western blot analysis. RNA FISH staining (FIG. 5) can be used to evaluate uptake of human GAS5 and MALAT1.

hASCexo treatment promotes wound healing and regeneration via (i) inhibiting apoptosis while promoting (ii) re-epithelialization (iii) neo-vascularization (iv) proliferation. To elucidate this, we propose immunohistological staining of wound sections. 8 μm thick longitudinal sections can be fixed with 4% paraformaldehyde. The sections will be visualized for the following: To evaluate apoptosis, sections can be stained for caspase-3 (total and cleaved) and cleaved PARP. Oxidative stress evaluated using antibodies for SOD1-3, GPx and 3NT. Staining with antibodies for CD31, VEGF, VEGFR and ang-2 as angiogenic markers. To evaluate infiltration of macrophages and response, sections can be stained for ED1+ (macrophages), CD20+ (B cells), CD3+ (T cells), inflammation markers IL1β, IL6, IL10, TNFα. To evaluate re-epithelialization, hematoxylin and eosin (H&E) staining can be performed and wound edges can be determined. Collagen deposit and organization of collagen deposition can be ascertained by Masson staining. The extra cellular matrix proteins (which are elevated upon wounding) can be evaluated with staining with antibodies for pro-collagen 1, collagen-1 and -3, fibronectin, tenascin. To evaluate neo-vascularization, increase in capillary density can be evaluated with CD34 staining. To visualize mature vessels, co-staining with CD34 with a-smooth muscle actin (α-SMA). To evaluate proliferation, cells can be stained with Ki67 and PCNA with DAPI for nucleus. The extent of staining can be quantified for percent proliferation. Cytokeratin (CK16) staining can be used to determine keratinocyte hyper-proliferation.

The wound healing process is accompanied with changes in gene expression that mediate the above processes. This study can elucidate lncRNA GAS5 and MALAT1 mediated mechanisms that promote repair and regeneration. To evaluate changes in gene expression in response to treatments, total RNA can be isolated and used in real time qPCR. This includes primers for GAS5, MALAT1, caspase 3, collagen 1 and collagen 3, elastin, PCNA, N-cadherin, fibronectin, VEGFR2, VEGFA, TGFβ, cyclin D1, MMP1, MMP9, IL-1β, IL-6, IL-8, IL-10, MCP1, NFkB, IKk, TNFα, GAPDH (control). Additionally, signaling pathways MAPK, AKT, PKC and inflammatory pathways mediated by TNFα and interleukins can be evaluated using automated western analysis (WES).

A key feature of wound healing is mitochondrial dysfunction which adversely affects cell death and survival, energy metabolism and intracellular signaling. To assess the response to hASCexo treatment, Agilent's Seahorse XF24 Analyzer can be used for the Mito Stress Test measuring oxygen rate consumption (OCR) and mitochondrial respiration. The wound sections are maintained at 37° C. throughout the procedure and the cohort can be simultaneously measured in the 24-well setup of Seahorse plates. Measuring the sections is advantageous as it considers the skin layers and their interplay.

It is highly beneficial to obtain a complete “omic” picture to translate this therapy to the clinic. RNAseq can be performed on days 7 and 21 treated with the hASCexo and hASCexo with GAS5 and MALAT1 levels manipulated (pooled cohorts to control for biological variances, M/F cohorts separately analysed). Previous experience with RNASeq that identified inflammation, cell bioenergetics and proliferation and signalling pathways in recovery post traumatic brain injury further underscores the importance of generating valuable transcriptomic data by RNASeq technology and linking it to long term recovery treatment of wounds with hASCexo. Validation of changes in RNA levels identified by RNA-seq can be achieved by qPCR, western blot, and in some cases IHC on tissue sections.

The pathways and specific genes up- or down-regulated with the treatments can be identified. Insulin signalling and GR-mediated inflammation pathway genes are expected to be dependent on GAS5 while apoptosis, proliferation, regenerative pathway genes to be dependent on MALAT1. Overlap and crosstalk of these pathways which culminate to promote repair and healing can be seen. Hence, pathway associations and Nodal assessments can be made on clustered and non-clustered gene sets using IPA, KEGG.

### C. Example 3: Long Noncoding RNA GAS5 Contained in Exosomes Derived from Human Adipose Stem Cells Promotes Repair and Modulates Inflammation in a Chronic Dermal Wound Healing Model

1. Introduction

Dermal wounds, which are breaks in the structure of skin due to cuts, lacerations, or incisional wounds post-surgery, are healed through a regulated repair process. At times, healing of wounds is delayed or slowed, resulting in chronic recalcitrant wounds. Underlying dermal inflammation is a substantial risk factor for impaired wound healing and often leads to chronic wound-related sequelae. Human adipose stem cells (hASCs) have shown tremendous potential in regenerative medicine. The stem cell antigens and markers of hASCs are similar to the mesenchymal stem cells isolated from the umbilical cord and bone marrow. hASCs have significant advantages over other sources of stem cells, primarily due to their abundance and easily accessible locations. The regenerative potential of hASCs is dependent on secreted bioactive material which is packaged and released in extracellular vesicles. This study aimed to improve the outcome of injury and promote wound healing by harvesting the secretome of hASCs for therapeutic intervention. The isolation of hASCs from adipose tissue has been mastered, where hASCs reside in the stromal vascular fraction. The stem cell antigens and markers have been characterized and evaluated for their renewal capacity and differentiation potential. It has been shown that the hASC secretome contains exosomes which are small extracellular vesicles (30-150 nm) that regulate fundamental cellular functions in recipient cells. While all cells secrete exosomes, their contents differ significantly. hASCs, their secretome collected as conditioned media (CM), and their exosomes have been characterized[1].

The advantages of the hASC exosome are that it provides a cell-free regenerative approach over other therapies, such as nanoparticles or stem cells. The natural biocomponents of exosomes are not toxic. It was previously demonstrated that an RNA component of exosomes drives its regenerative therapeutic ability. The presence of several long noncoding RNAs (lncRNA) in the secretome of hASCs, and additionally these studies indicated specific lncRNAs that were packaged in large amounts in exosomes [1-3]. The lncRNAs are packaged into exosomes to prevent their degradation by nucleases. Exosomes release the lncRNA cargo into the target cells where it regulates gene expression and influences the genomic landscape.

Here, the outcome of wound healing with treatment with hASC exosomes was evaluated using human dermal fibroblasts (HDF). The results showed a significantly accelerated wound closure time, supporting the use of hASC exosomes in dermal wound healing. Previously, using RNAseq to analyze the lncRNA content of hASC exosomes, the results identified lncRNA growth-arrest specific-5 (GAS5) and metastasis-associated lung adenocarcinoma transcript 1 (MALAT1), which were highly enriched in exosomes [1,4]. In prior studies, MALAT1 was evaluated as it is a strong regulator of expression of several genes. MALAT1 accounted for at least 50% of wound healing by exosomes and the pathways and alternative splicing events were identified affected by MALAT1 contained in exosomes [2,5]. However, the role of GAS5 contained in hASC exosomes in wound repair is not yet deciphered. Hence, whether exosomal GAS5 was essential for wound repair was determined. Further, wound infection is a significant clinical problem and thus we evaluated wound healing under lipopolysaccharide (LPS)-induced chronic inflammation and the efficacy of treatment with hASC exosomes. Using ingenuity pathway analysis (IPA), the inflammation signaling pathway mediators and Toll-like receptors that were implicated in wound healing by hASC exosomes under conditions of chronic low-grade inflammation were identified. Further, the genes whose expression changed in a GAS5-dependent manner were identified. These results elucidate the mechanisms by which hASC exosomes promote healing in chronic wounds.

### 2. Materials and Methods

i. Cell Culture and Treatments

Primary human dermal fibroblasts (HDF) were purchased from Sciencell (catalog #PCS-201-012) and passaged as preconfluent cultures in fibroblast media (Sciencell catalog #2301). Human adipose stem cells (hASC) were purchased from Zenbio (catalog #ASC-F) and grown to confluency in its media (Zenbio, catalog #PM-1; also called stem cell media as it maintains the stemness of hASC). The hASCs were characterized by detecting stem cell antigens and markers (positive for CD44, CD90, CD115 and negative for CD31, CD45, CD14) using flow cytometry as described by us in our prior publication [1]. The hASC were cultured up to 2 passages to obtain exosomes from its secretome to evaluate exosomes as a therapeutic for treatment of dermal wounds. All cells were grown at 37° C. and 10% CO2. To induce inflammation, LPS was added to cells [2-4]. For acute treatment experiments, 5 ng/mL LPS (Sigma) was added to 90% confluent HDF for 6 h and then medium was changed to remove LPS. For chronic treatment experiments, 5 ng/mL LPS was added to 90% confluent HDF for 6 h to induce inflammation. LPS was maintained in medium, and cells were treated with or without hASC exosomes for 4 days, as indicated in the experiments. Oxidative stress is a key player in the pathogenesis of non-healing wounds. Then, 100 μM of H2O2 (Fisher catalog #H323-500) was added for 1 h to HDF cells to induce oxidative stress, after which the medium was changed to remove H2O2 and the cells were treated with or without hASC exosomes.

ii. Exosome Isolation from Human Adipose Stem Cells (hASCs)

The hASC media (Zenbio, catalog #PM-1) was centrifuged at 100,000×g for 60 min to remove extracellular vesicles and exosomes from media. The hASCs at 90% confluency were then cultured in exosome-free hASC media to ensure that all exosomes isolated were derived from hASCs. After 48 h, the conditioned medium (CM) from 8×10⁶ hASC was collected. Exosomes were isolated from CM as previously described by our lab [1, 5]. Briefly, conditioned media derived from hASCs was collected after 48 h and centrifuged at 3000×g for 15 min to remove dead cells. ExoSpin™ (Cell Guidance system; Catalog EX05) reagent was added to the CM and incubated for 20 min at room temperature. Following centrifugation at 1500×g for 30 min to remove cellular debris, the supernatant was applied to the top of ExoSpin columns and centrifuged at 50×g for 60 s. Exosomes were eluted in PBS by centrifugation at 50×g for 60 s. Nanoparticle tracking analysis with NanoSight (NTA3.1, Build 3.1.46 RRID SCR-014239) was used to analyze peak diameter and the concentration of exosomes obtained from 106 hASC. Analysis showed exosome size to be 94±7 nm.

iii. Transfection of hASCs

To label hASC exosomes, 1×10 6 hASCs (Zenbio, catalog #ASC-F) were trypsinized and cell pellets were transfected with either 2 μg mCherry (Addgene, 128744) or 2 μg GFP-pmax (included in Nucleofector kit) using a Nucleofector® kit (Lonza, catalog #VPE-1001). The cell/DNA solution was transferred to a cuvette and the program initiated (0.34 kV, 960 pf). Medium (500 μL) was added immediately and cells were gently transferred to 100 mm plates and allowed to grow for 48 h. Exosomes were verified to contain mCherry by PCR using sense primer 5′-CAGGACGGCGAGTTCATCTA-3′ and antisense and verified to contain GFP by PCR using sense primer 5′-AGGCGTGTACGGTGGGAG-3′ and antisense 5′-CTACAAATGTGGTATGGCTGA-3′. To deplete MALAT1 from hASC exosomes, 1 μM MALAT1 antisense oligonucleotide (ASO; ID: 39524 ASO from Ionis Pharmaceuticals, validated for specificity and designed for efficient uptake by cells as demonstrated by us previously [5]), was added to the hASCs and incubated for 48 h. To deplete GAS5 from hASC exosomes and from HDF, 25 nM GAS5 siRNA (ThermoFisher/Ambion catalog #n520782) was transfected into cells using RNAiMax (ThermoFisher catalog #13778075) for 48 h. The siRNA, selected from four siRNAs that target separate areas on GAS5 and evaluated for optimal knockdown of expression of GAS5, was validated for efficacy, non-toxicity, and specificity to eliminate off-target effects, as described by us previously [5-7]. A negative control siRNA (Thermo Fisher catalog #4404021), RNAiMax alone (transfection control) and an untreated control were included in all experiments. The expression levels and knockdown of MALAT1 and GAS5 in the exosomes were verified using human MALAT1 and GAS5 primers in qPCR and absolute levels were determined as described below.

iv. Real-Time Quantitative Polymerase Chain Reaction (RT-qPCR)

Total RNA was isolated from 1×106 HDF using Trizol™ (Thermo Fisher Scientific) as per the manufacturer's instructions. A quantity of 1 μg of RNA (260/230>1.8 and 260/290>1.8) was used to synthesize cDNA using ReadyScript™ synthesis mix (Sigma RDRT). Real-time qPCR was then performed in triplicate using 1 μL of cDNA and Maxi-ma SYBR Green/Rox qPCR master mix (Thermo Scientific). Amplification was performed on the ViiA 7 (ABI). Primers were purchased from Origene (qSTAR qPCR primer pairs). These primer pairs were pre-designed, validated, and tested for specificity to eliminate off-target effects. Primers had a Tm of 60-61 and an amplicon of 95-140 bp. The optimal primer concentration was determined from a range of 50-900 mM. The final concentration of each primer pair was selected to ensure efficiency and specificity for its target in HDF based on the dissociation curve that showed a single, sharp peak indicating that the primers amplify one specific target (described by us previously in [8].) Primers used in qPCR included GAS5 S 5′-CTTCTGGGCTCAAGTGATCCT-3′, GAS5 AS 5′-TTGTGCCATGAGACTCCATCAG-3′, MALAT1 S 5′-CTTCCCTAGGGGATTTCAGG-3′, MALAT1 AS 5′-GCCCACAGGAACAAGTCCTA-3′, IL1β S 5′-CCACAGACCTTCCAGGAGAATG-3′, IL1β AS 5′-GTGCAGTTCAGTGATCGTACAGG-3′, IL6S 5′-AGACAGCCACTCACCTCTTCAG-3′, IL6 AS 5′-TTCTGCCAGTGCCTCTTTGCTG-3′, TLR4 S 5′-CCCTGAGGCATTTAGGCAGCTA-3′, TLR4 AS 5′-AGGTAGAGAGGTGGCTTAGGCT-3′, TLR7 S 5′-CTTTGGACCTCAGCCACAACCA-3′, TLR7 AS 5′-CGCAACTGGAAGGCATCTTGTAG-3′, IR S 5′-GTTTTCGTCCCCAGGCCATC-3′, IR AS 5′-CCAACATCGCCAAGGGACCT-3′, ITGB2 S 5′-AGTCACCTACGACTCCTTCTGC-3′, ITGB2 AS 5′-CAAACGACTGCTCCTGGATGCA-3′, IL18 S 5′-GATAGCCAGCCTAGAGGTATGG-3′, IL18 AS 5′-CCTTGATGTTATCAGGAGGATTCA-3′, CCL17 S 5′-TTCTCTGCAGCACATCCACGCA-3′, CCL17 AS 5′-CTGGAGCAGTCCTCAGATGTCT-3′, and GAPDH S 5′ GATCATCAG-CAATGCCTCCT-3′ and GAPDH AS 5′-TGTGGTCATGAGTCCTTCCA-3′. For absolute quantification using SYBR Green qPCR, a standard curve was generated for each gene in every assay. For this, 100-0.4 ng of RNA from HDF was reverse-transcribed as described above. The resulting cDNA was used to obtain a standard curve correlating the amounts with the threshold cycle number (Ct values). A linear relationship (r2>0.96) was obtained for each gene. Plate set up included the standard series, no template control, and no re-serve transcriptase control. The dissociation curve was analyzed for each sample. Absolute quantification (AQ) for expression levels of individual transcripts was calculated by normalizing the values to GAPDH. Relative quantity (RQ) was determined by the ΔΔCt method with GAPDH as the endogenous control and control sample as the reference calibrator. In addition, to validate the qPCR data, 1 μL of cDNA was amplified with the primer pairs, including GAPDH endogenous control primers, using JumpStart ReadyMix (Sigma P0982); products were run on a 1% agarose gel and stained with ethidium bromide for visualization of bands and imaged in ProteinSimple FluorChem M. GAPDH stability under different treatments was verified by visualizing a single band per sample followed by densitometric analysis of the bands across treatments using the integrated Al-phaView® software 3.5.0 (ProteinSimple, San Jose, California).

Droplet digital PCR (ddPCR): mRNA copy counts of GAS5 and MALAT1 in 1 μg exosome were determined by ddPCR which is housekeeping gene independent absolute copy number detection. RNA was isolated from 1 μg exosomes and entire quantity was used in generating cDNA. 2 μl cDNA was used in EvaGreen Super mix (BioRad) and loaded with ddPCR oil in cartridges and plate sealed with foil on PX1 as per manufacturer's instructions. After droplet generation in QX100, droplet emulsions were transferred to 96-well plates and amplified in BioRad ddPCR thermocycler and results were read on QX200 Droplet reader. Data was analyzed using QuantSoft Analysis Pro (1.0.596) to generate copy number.

v. Western Blot Analysis

Protein lysates were obtained from the hASC exosome preparations using lysis buffer containing protease inhibitors (Cell signaling #9803s). The bicinchoninic acid (BCA) protein method was used to quantitate total protein in the samples. A quantity of 40 μg of lysates were separated by SDS-PAGE using 8-15% acrylamide gels, then electrophoretically transferred to nitrocellulose membranes (at 90 V for 1 h), blocked with 5% bovine serum albumin in Tris-buffered saline containing 0.1% Tween 20 (TBST) for 1 h. Membranes were washed thrice with TBST. To detect the presence of tetraspanins (mark-ers for exosomes), the membrane was incubated overnight at 4° C. with either anti-CD9 (Abcam ab236630; 1:1000), anti-CD63 (Abcam ab134045; 1:1000), or anti-CD81 (Abcam ab79559; 1:1000). After incubation with anti-rabbit IgG-HRP (1:3000) for 30 min, enhanced chemiluminescence (Pierce) was used for detection and images were digitally captured using ProteinSimple FluorChem M. Experiments were repeated thrice, and representative bands demonstrating the presence of CD9, CD63, and CD81 on exosome preparations are shown in FIG. 1a. Densitometric analysis was performed using the AlphaView® software 3.5.0 (ProteinSimple, San Jose, California).

vi. Inflammation Array and Analysis

Total RNA was extracted from HDF cells and cDNA was generated as described. The cDNA was amplified using the human inflammation array (Qiagen catalog #PAHS-077Z) according to the manufacturer's instructions. The array consists of 90 inflammation pathway genes, housekeeping genes, and positive and negative controls. The data were analyzed using Qiagen/GeneGlobe's Enterprise Data Analysis Solutions and differentially expressed genes compared to controls were identified. Data were further analyzed using ingenuity pathway analysis (IPA) to identify direct and indirect relationships and a comparison analysis was performed to identify causal networks and signaling pathways. Results were filtered to human fibroblast data and z-scores ≥±2 were considered.

vii. Seahorse Metabolic Assay

HDF cells were plated into a poly-D-lysine-coated Seahorse XFp cell culture mini-plate (Agilent Technologies, CA, USA) at a density of 4000 cells per well as deter-mined by optimization cycles. To mimic oxidative stress in HDF and the response to treatment with exosomes, HDF were treated acutely with 100 μM H2O2 for 1 h followed by a change of cell culture medium and treatment with 1 μg of exosomes (Exo) or GAS5-depleted exosomes (Exo-G5) for 18 h. The media was then changed to Seahorse XF Media (supplemented to 100 mM pyruvate, 200 mM glutamine, and 2.5 M glucose) before being imaged on the Keyence microscope to calculate cell count for normalization. Mitochondrial function in live cells was determined by performing the Cell Mito Stress Test [9] using Seahorse XF (Agilent). The plate was incubated in a non-CO2 incubator at 37° C. for 1 h. Seahorse sensor cartridges were prepared, and solutions were loaded into ports as described for the XFp Mito Stress Test (100 μM oligomycin, 100 μM fluoro-carbonyl cyanide phenylhydrazone FCCP, and 50 μM antimycin A/rotenone were added to cells. The experiment was performed using the Seahorse XFp Analyzer. Oxygen consumption rates were measured at intervals of approximately 5-8 min. The measurements were normalized to cell counts and data were analyzed using the Agilent Wave software.

viii. AOPI Cellular Survival Assay

HDF cells were grown in a 12-well plate and at 90% confluency treated with 5 ng/mL LPS for 6 h followed by exosome treatment, as described in experiments. Cells were then trypsinized and washed once with PBS. The cell pellet (containing one million cells) was resuspended in 500 μL PBS and fixed by slow, drop-wise addition of 4.5 mL ice-cold 70% ethanol while gently vortexing. Samples were incubated overnight at 4° C. to complete fixation and then stored at −20° C. until stained. A fresh solution of propidium iodide (1 mg/mL, ViaStain™ CS1-0109) and RNase A (2 mg/mL, Thermo Fisher EN0531) was diluted in water. Fixed cells were centrifuged at 1000 rpm for 5 min. The cell pellet was washed twice with PBS and the pellet was resuspended in 50 μL PI/RNase A solution and incubated at room temperature for 5 min. One milliliter PBS was added, and samples were divided to create unstained negative control for analysis. Acridine orange (ViaStain™ CS2-0106) was added (1:1) to samples for staining and incubated at 37° C. for 30 min then analyzed on the Nexcelom K2 cellometer.

ix. Cell Migration by Scratch Assay

To evaluate the efficacy of hASC exosomes in dermal wound healing, we performed the scratch assay, a widely used wound healing assay, in HDF. HDF cells were grown to confluency in 35 mm dishes with p-Dish inserts (Ibidi solutions™, 81176) to make consistent and reproducible 500 μm gaps. Cells were treated with hASC exosomes, as described in each experiment, then the inserts were removed and cell migration was imaged on the Keyence BZx-810 microscope at 4× magnification. Images were taken at the same location saved into the Keyence BZx-810 microscope with images taken at 0 and 18 h or 0, 18, and 24 h, as indicated in the individual experimental setup. The open areas between lateral cell boundaries were quantified using Image J software with the plugin Wound_healing_size_tool.

x. Three-Dimensional Wound Assay

To mimic the microenvironment of skin, a 3D wound healing model was used. A collagen scaffold was prepared in 35 mm plates. To do so, for each well, 800 μL of collagen type I (Sigma cat #C3867) was reconstituted with 100 μL ice cold 10×DMEM (+Phenol Red) and mixed by slow and gentle pipetting on ice. Color change of DMEM was observed from red to yellow, indicating the acidity of the solution. Then, 10× reconstitution buffer was made fresh (2.2 g of sodium bicarbonate and 4.8 g of HEPES in 100 mL of distilled water, filtered through a 0.22 μm vacuum filter). A quantity of 100 μL of 10× reconstitution buffer was gently mixed into collagen/DMEM solution. Color change of DMEM was observed from yellow to light pink, and pH was verified to be within the range of 7.1-7.4. The mixture was incubated on ice for 5 min, then collagen was spun at 10,000 rpm 3 min at 4° C. to remove air bubbles. Collagen was pipetted into a 24-well plate and permitted to polymerize at 37° C. for 2 h. HDF cells were plated at 300,000 cells per well and grown to confluency. A circular wound was generated using a cut pipet tip punched into collagen. The cavity was filled with collagen to generate a 3D circular wound free of cells. The 3D wound model was kept at 37° C. The wound gap was imaged using Keyence BZx-810 every 24 h. Wound closure was calculated each day using Keyence's cell migration assay with Hybrid cell count software to calculate wound gap area.

xi. Immunocytochemistry

HDF cells were plated into a 12-well plate, lipopolysaccharide (LPS; 5 ng/mL) was added for 6 h, and the medium was changed to remove LPS and 1 μg Exo or Exo-G5 was added for 18 h. Cells were fixed with 4% paraformaldehyde for 30 min at room temperature and blocked with 1% bovine serum albumin in PBS for 1 h. Cells were incubated overnight in primary antibody against Ki67 (1:1000, Novacastra). Cells were then washed with PBS and rocked in the dark for 1 h in Alexafluor 488 secondary antibody (1:1000, Invitrogen). Cells were rinsed with PBS thrice for 30 s each and stained with DAPI mounting media. Images were captured using a Keyence BZx-810 microscope and analyzed using Keyence Analyzer software.

xii. Statistical Analysis

All experiments were repeated 3-5 times as biological replicates and experimental samples run in triplicate to ensure the reproducibility of results. Analyses were performed using PRISM™ software and analyzed using a two-tailed Student's t-test and one-way or two-way ANOVA, as indicated in figure legends. * p<0.05, ** p<0.01, and *** p<0.001 were used as significant measures.

### 3. Results

i. Exosomes from hASCs Promote Wound Healing

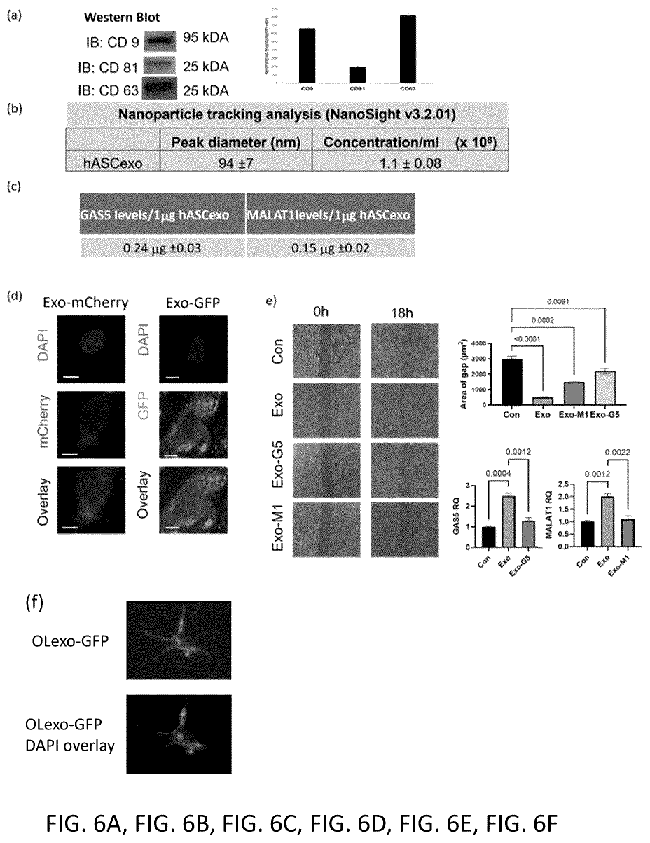

Human adipose stem cells (hASCs) and their secretome show tremendous potential in healing wounds. We sought to evaluate the role of the smallest nanovesicles called exosomes secreted by hASCs. Exosomes were isolated from the conditioned media of hASCs. Western blot analysis (FIG. 6A) was performed to validate the presence of the tetraspanin markers of human exosomes CD9, CD63, and CD81. To evaluate the purity, concentration, and size of exosomes, NanoSight and its analysis software NTA was used (FIG. 6B). Our previous studies demonstrated that long noncoding RNAS (lncRNAs) contained in hASC exosomes are crucial to the regenerative properties of exosomes. Using RNAseq and qPCR, our results had shown that amongst the noncoding RNAs, the lncRNAs GAS5 and MALAT1 were highly enriched in hASC exosomes [1]. Hence, using qPCR we deter-mined the levels of GAS5 and MALAT1 in every batch of hASC exosomes used in these experiments. FIG. 6C shows the expression levels of GAS5 and MALAT1 per microgram of exosomes and the tolerance ranges of levels of GAS5 and MALAT1 contained in the preparations of hASC exosomes.

To determine uptake of hASC exosomes by human dermal fibroblasts (HDF), 1 μg of exosomes labeled with either mCherry or GFP Amax was added to HDF for 24 h. The cells were then imaged using a Keyence microscope. The results (FIG. 6D) demonstrates that hASC exosomes are efficiently taken up by HDF and are equally distributed within the cytoplasm and nucleus.

Next, we sought to evaluate the efficacy of hASC exosomes in dermal wound healing and further determine whether MALAT1 or GAS5 contained in the hASC exosomes affected the ability of hASC exosomes to promote the repair of wounds. To do so, we performed the scratch assay, a widely used wound healing assay, in HDF. GAS5 or MALAT1 was depleted from hASC, as described in the section on Materials and Methods, and exosomes were isolated from the conditioned media. HDF cells were plated in 35 mm dishes with p-inserts to create scratches (cell-free gaps) mimicking dermal wounds. After 24 h, the insert was removed and HDF were treated with 1 μg of hASC exosomes (Exo), GAS5-depleted exosomes (Exo-G5), or MALAT1-depleted exosomes (Exo-M1), as indicated in the experiments. Images were taken using a Keyence BX810 microscope at 0 h and 18 h. Quantification of wound healing was achieved by ACAS image analysis software (Ibidi solutions™). Our results (FIG. 6E) demonstrate that exosomes closed the wound gap 84% faster compared to control. Compared to exosomes treatment (Exo), depleting GAS5 (Exo-G5) significantly reduced the wound healing outcome, thereby lowering the efficacy of exosomes. Exo-M1 treatment also decreased healing in concurrence with our previously published data [10]. These results demonstrate that exosome treatment accelerated wound closure and, further, that depleting either GAS5 or MALAT1 from exosomes substantially attenuated exosome-mediated wound healing. Our previous studies have demonstrated the role of MALAT1. In this project, we focused on elucidating the role of GAS5 contained in hASC exosomes in dermal wound healing.

ii. hASC Exosome Treatment Alleviates Oxidative Stress in HDF

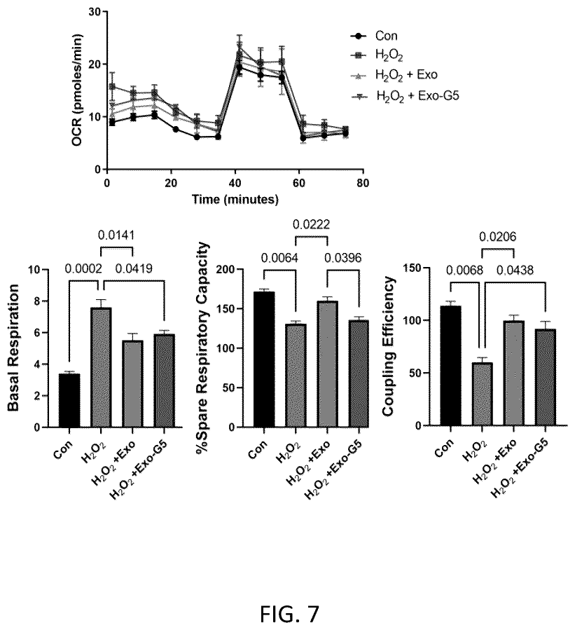

Low concentrations of reactive oxygen species are integral to wound healing in the skin as they aid to fight any invading microorganisms and promote cell survival pathways. However, oxidative stress is produced by high levels of reactive oxygen species, such as H₂O₂. Oxidative stress is a key player in the pathogenesis of non-healing wounds. To mimic oxidative stress in HDF and the response to treatment with exosomes, HDF were treated acutely with a high concentration of H₂O₂. A quantity of 100 μM of H₂O₂ was added to HDF for 1 h, followed by change in cell culture medium and treatment with 1 μg of exosomes (Exo) or GAS5-depleted exosomes (Exo-G5) for 18 h. Mitochondrial function in live cells was determined by performing the Cell Mito Stress Test [9] using Seahorse XF (Agilent). Results (FIG. 7) showed that H₂O₂ treatment increased basal oxygen consumption rate (OCR) due to an increase in proton leakage along with decreased respiratory capacity and decreased coupling efficiency. Treatment with Exo significantly reversed the detrimental effects of H₂O₂ on OCR, while treatment with Exo-G5 showed a lower ability to reverse the damage caused by H₂O₂ compared to Exo.

iii. GAS5 Contained in hASC Exosomes is Critical for Regeneration in Wound Models

Next, the effect of wound healing outcomes under inflammatory conditions were evaluated with exosome treatment. To mimic acute inflammation, lipopolysaccharide (LPS; 5 ng/mL) was added for 6 h, medium was changed to remove LPS, and 1 μg Exo or Exo-G5 was added for 18 h. This experimental setup was utilized in the following series of evaluations of the efficacy of hASC exosomes in wound healing.

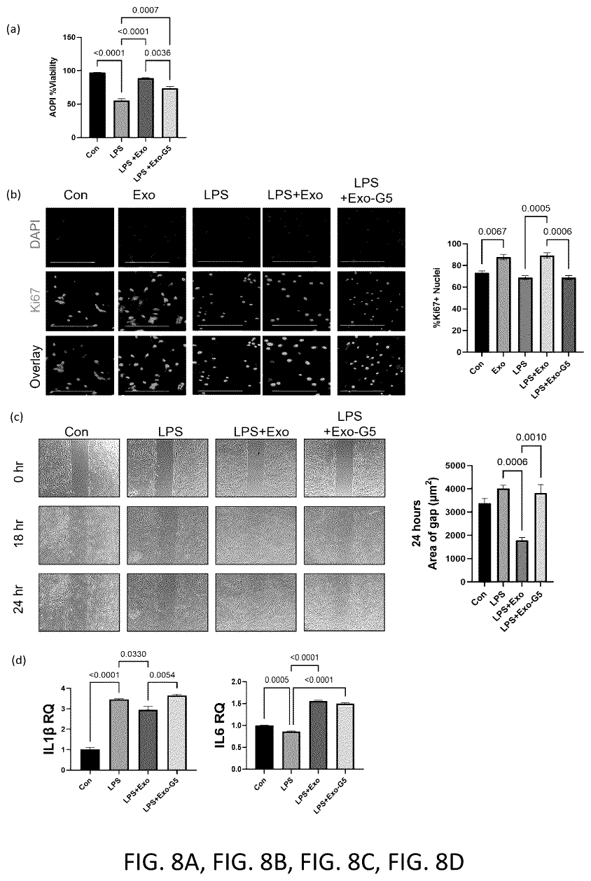

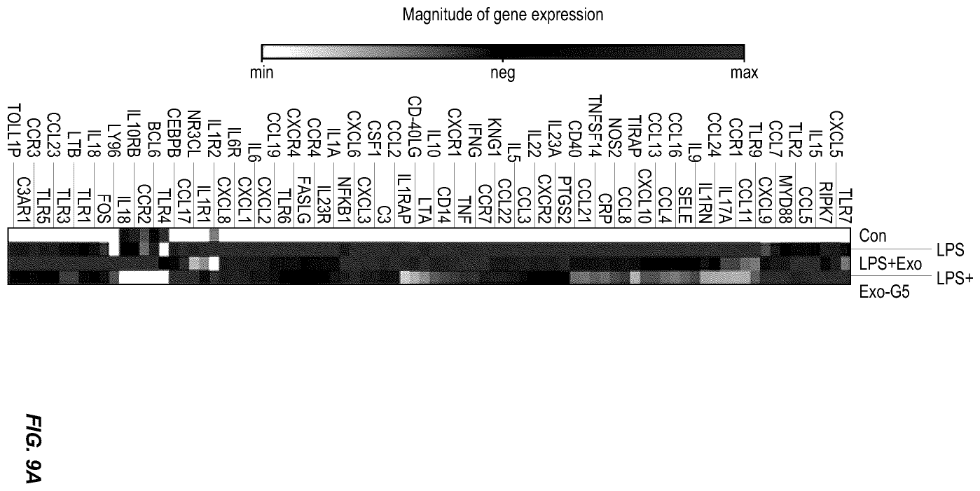

Cell viability was determined using the AOPI assay in the above experimental setup. Results (FIG. 8A) show that LPS decreased cellular viability. Treatment with exosomes improved cell viability, while GAS5-depleted exosomes attenuated this effect.

Next, cellular proliferation was evaluated using immunocytochemistry staining for Ki67 in the experimental setup described above. Results (FIG. 8B) showed a significant decline in proliferation with LPS treatment, which was rescued by treatment with exosomes in a GAS5-dependent manner.

Wound closure was evaluated using the scratch assay in an LPS-induced inflammation environment in the experimental setup described above. A scratch assay was performed as described in Materials and Methods. Results (FIG. 8C) show that LPS-induced inflammation hinders wound healing measured as closure of gap. Treatment with hASC exosomes (Exo) accelerated closure of gap, while depletion of GAS5 (Exo-G5) significantly attenuated the effect of hASC exosome-mediated repair in an inflammatory environment. Finally, in the experimental setup described above, total RNA was extracted and SYBR Green real-time qPCR was performed to evaluate the levels of IL1β and IL6, which are markers of inflammation. Results (FIG. 8D) showed that LPS increased expression of IL1β and IL6, treatment with hASC exosomes decreased its levels, while exosomes depleted of GAS5 were unable to decrease LPS-induced increase in IL1β and IL6 levels.

iv. GAS5 Contained in hASC Exosomes Modulates Inflammation in a Chronic Wound Model

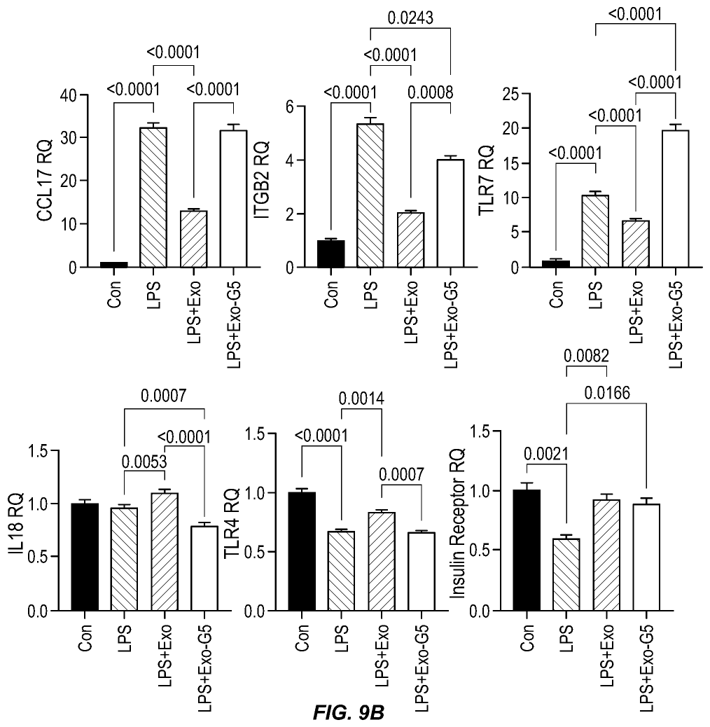

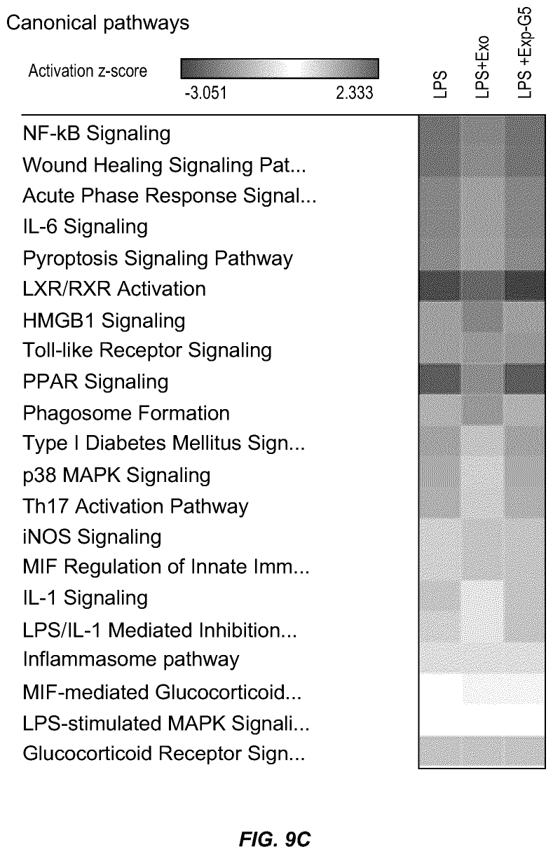

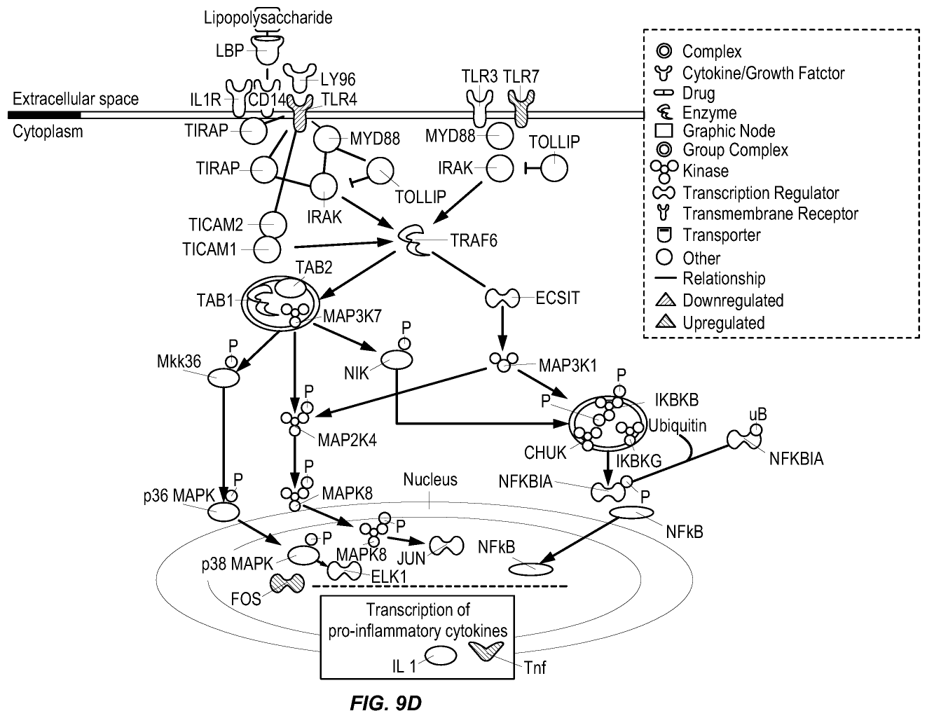

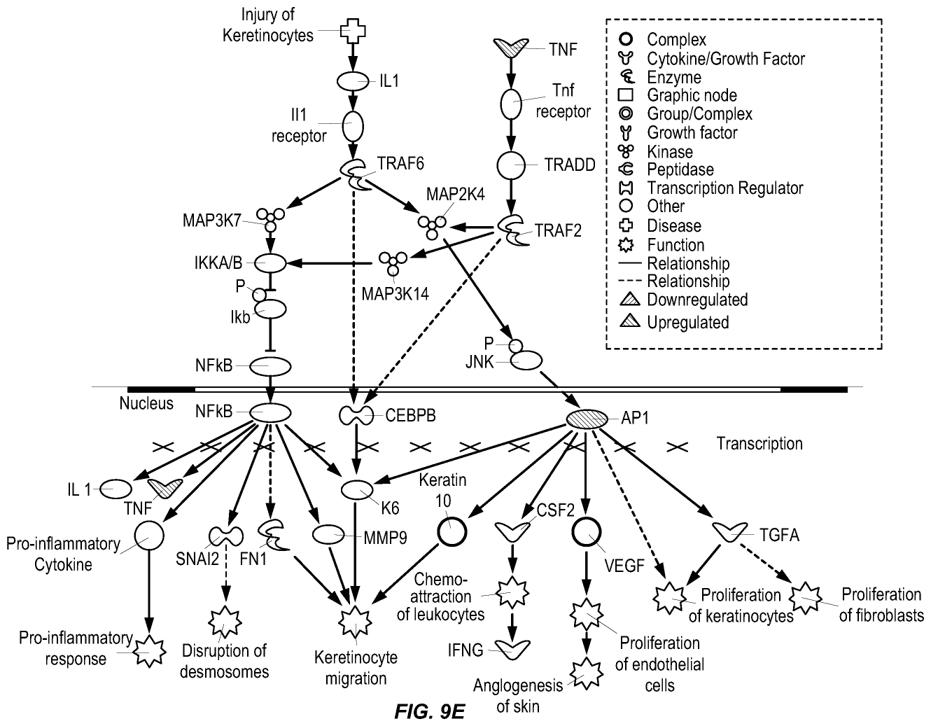

At times, the healing of wounds is delayed or slowed, resulting in chronic recalcitrant wounds. To mimic this chronic inflammation scenario in vitro, HDF cells were pre-treated with low dose (5 ng/mL) LPS for 6 h to induce inflammation. After initiation of inflammation, cells were then treated with either Exo or Exo-G5. LPS (5 ng/mL) was maintained in the medium to mimic chronic low-grade inflammation, along with the exosome treatment for these cells for 4 days. Cells were harvested after 4 days, and RNA was isolated. To identify the genes involved in inflammation pathways that were affected with the chronic LPS treatment, the human inflammatory response array (Qiagen PAHS-077ZA) was used. FIG. 9A shows the differentially expressed genes compared to control samples. The results showed that most genes in the inflammatory pathway, such as those of the Toll-like receptor (TLR) family and chemokine (C-C motif and C-X-C motif) ligands and receptors and interleukins (IL), were upregulated at the end of 4 days of LPS treatment; however, some genes were downregulated at this timepoint. Significantly, TLR4 was downregulated by 33% after 4 days of low-concentration LPS treatment.

Next, genes that were affected by exosomes in a GAS5-dependent manner in the chronic LPS-induced inflammation environment were analyzed for. Towards this, the pattern for genes whose expression compared to control changed with LPS was analyzed; treatment with exosomes (Exo) reversed the LPS-induced changes, and further depletion of GAS5 in exosomes (Exo-G5) attenuated the changes of Exo in LPS samples. When pattern 1 was compared to control, the genes showed upregulation with LPS, downregulation with Exo, and upregulation with Exo-G5. The top genes with statistical significance that follow pattern 1 are TLR7, CCL17, and ITGB2. When pattern 2 was compared to control, the genes showed downregulation with LPS, upregulation with Exo, and downregulation with Exo-G5. The top gene with statistical significance that follows pattern 2 is TLR4. The genes identified in patterns 1 and 2 were then confirmed using real-time qPCR (FIG. 9B). Since a previous study demonstrated that GAS5 regulated expression of the insulin receptor (IR), IR levels were also evaluated, and qPCR results showed that IR expression followed pattern 2.

The array data were further analyzed using ingenuity pathway analysis (IPA), and FIG. 9C shows that the top canonical pathways, including NF-kB signaling, wound healing signaling, acute phase response, and IL6 signaling, were changed in a GAS5-dependent manner. Since our results showed that TLR4 and TLR7 levels were changed in a GAS5-dependent manner, analysis was then performed to integrate the genes and their pathways. FIG. 9D shows the intracellular location of the TLR and NF-kB pathway and the genes that were significantly affected while comparing Con vs. LPS vs. LPS+Exo vs. LPS+Exo-G5, including TLR7, TLR4, NF-kB, interleukins, and cytokines. To understand how changes in expression levels of these genes affected wound healing, network analysis was performed and the results showed how NF-kB, cytokines, and interferons integrate to promote wound healing in the skin. Overall, the data analysis showed the integration of the response to chronic LPS treatment wherein LPS binds to the TLR4 receptor along with changes in expression of TLRs which recruit cofactors and activate transcription factors and ultimately result in the expression of interferons and cytokines.

v. Depletion of GAS5 in HDF Cells Increases Expression of Toll-Like Receptor 7

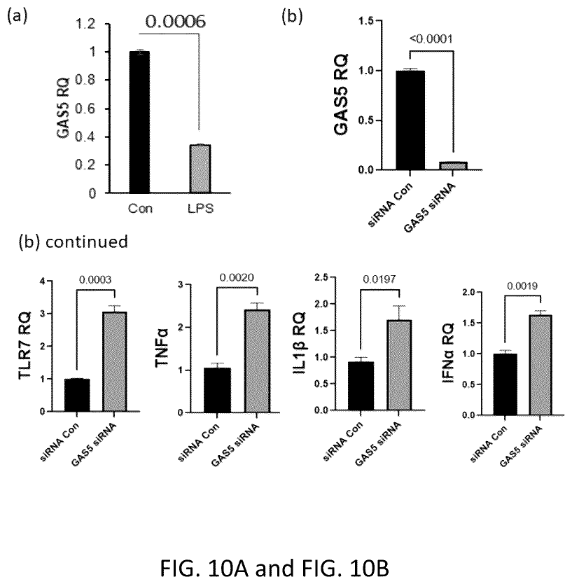

The results from that array showed that CCL17, ITGB2, and TLR7 were significantly increased in chronic LPS-induced inflammation. It was evaluated whether chronic LPS treatment in HDF affected GAS5 levels. HDF cells were treated with LPS (5 ng/mL) for 4 days and real-time qPCR was performed using primers for GAS5. Results (FIG. 10A) showed that LPS-induced chronic inflammation resulted in a 65% decrease in GAS5 levels in HDF. Hence, it was evaluated whether siRNA-mediated depletion of GAS5 in HDF directly affected expression of either CCL17, ITGB2, or TLR7. GAS5 was depleted by transfecting 25 nM GAS5 siRNA (see Materials and Methods), and real-time qPCR results (FIG. 10B) demonstrate that GAS5 depletion significantly increased TLR7. CCL17 and ITGB2 levels did not change in a significant manner (not shown). Further, the qPCR results demonstrated that the levels of IFNα, IL1β, and TNFα increased with depletion of GAS5 in HDF

vi. GAS5 Contained in hASC Exosomes Mediates Repair Post-Injury in a Chronic Wound Model

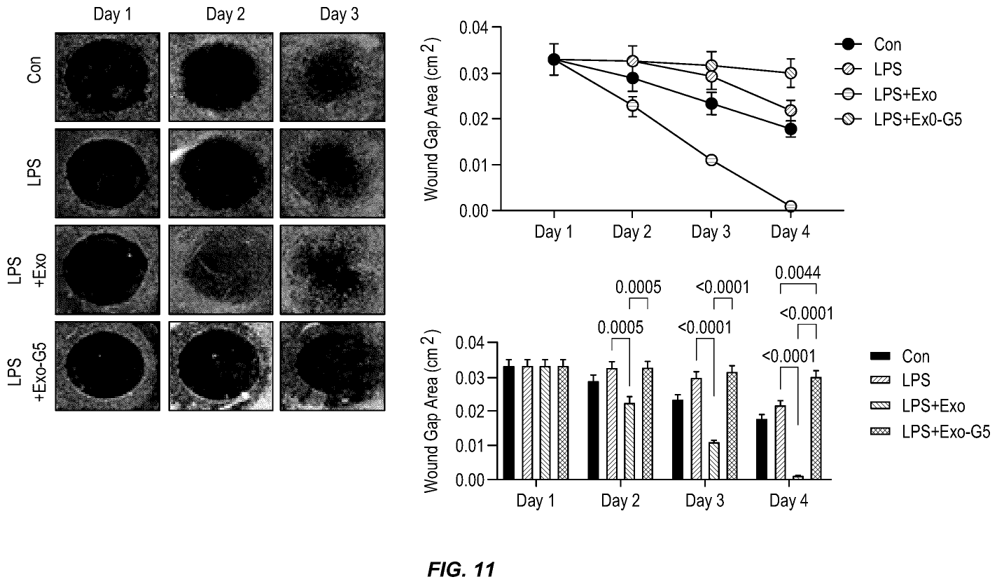

To mimic the microenvironment of skin, a 3D wound healing model was used (described in Materials and Methods). After initial pre-treatment with LPS (6 h), cells were treated with Exo or Exo-G5. LPS was maintained in the medium along with the Exo or Exo-G5 treatment for these cells for 4 days to mimic accompanying chronic low-grade inflammation. Results (FIG. 11) showed that chronic low-grade inflammation induced by LPS hinders the closure of wounds. Further, Exo treatment significantly accelerates wound closure compared to control in a chronic inflammatory environment. Exo-G5 treatment significantly attenuated wound closure.

### 4. Discussion

Exosomes are secreted by human adipose stem cells (hASCs) and are important mediators of the repair and regeneration post-injury attributed to hASCs [7, 10-14]. Exosomes from hASCs are thus referred to as a stem cell-based, cell-free therapy for wound healing. The cargos of hASC exosomes contain a number of proteins necessary for targeting cells and mediating uptake and recycling, while the RNA cargos modulate gene expression in target cells. It was previously demonstrated that the noncoding RNA content of Exo drives the repair and regeneration of the Exo [7]. The previous studies in HDF and HT22 cells and in vivo models of traumatic brain injury have demonstrated the role of MALAT1, contained in hASC exosomes, in neuronal wound healing and showed that it accounted for a portion of the regenerative properties of hASC exosomes [5,6,12].

Other studies have identified miRNAs that modulate wound healing [15-17]. The results presented here demonstrated that lncRNA GAS5 was highly enriched in hASC exosomes, but its function is not yet determined. Hence, in this project the focus was on elucidating the role of GAS5 contained in hASC exosomes in dermal wound healing. The role of lncRNA GAS5 in promoting wound healing was evaluated and further elucidated the pathways affected in wound healing with an underlying chronic inflammation in human dermal fibroblasts.

To control for batch-to-batch variances, we strictly adhered to our standardization procedure. Multiple vials of hASCs from a pooled donor lot were established at the same time and served as our master cell bank and the entire project was performed using these hASCs. We carefully controlled for the stem cell markers and contents, as described in FIG. 1. Such rigid adherence is necessary to translate the therapy into clinic using GMP facilities. Wounds are healed by a highly regulated process which involves the initial response of inflammation followed by proliferation and remodeling. Exosomes from hASCs (Exo) function to deliver their cargos to recipient cells and thereby affect the genomic landscape to promote healing. Hence, we undertook cellular, genetic, and physiological wound healing in vitro assays to understand the mechanisms involved in the healing of wounds with Exo treatment.

Using the scratch assay as a model for in vitro wounds, it was demonstrated that exosomes depleted of GAS5 (Exo-G5) were significantly hindered in their ability to close the wound gap compared to the Exo treatment. Other studies have identified endogenous GAS5 as pivotal in wound healing in diabetic wounds [18]. It was demonstrated that GAS5 expression is significantly lower in type 2 diabetes and further demonstrated that GAS5 regulates the expression of insulin receptor [6, 19]. Other studies have shown that application of insulin to the wound also promoted healing [20-22]. Kino et al. showed that GAS5 regulated glucocorticoid receptor (GR) target genes by sequestering GR [23]. These studies shed light on the multifaceted role of GAS5 in regulating several signaling cascades depending on the particular cell type and environment.

In physical injury and infection, there is an immediate increase in inflammation which is critical for response and the initiation of healing. As healing progresses, inflammation is resolved and pathways mediating repair and proliferation are initiated [24, 25]. Diseases such as diabetes, cardiovascular diseases, and obesity are accompanied by chronic low-level inflammation, which is a substantial risk factor for impaired wound healing and often leads to chronic wound-related sequelae. Delayed or slow wound healing is a significant problem in the clinic. Amongst the risk factors promoting recalcitrant wound healing is underlying infection in which wounds exhibit chronic low levels of inflammation. In this study, the HDF were treated with a low dose of LPS to mimic the chronic low inflammation that accompanies certain diseases [26]. LPS, usually on Gram-negative bacteria, is commonly associated with wound infections [27, 28]. The innate immune system is activated as a defense mechanism against microbial infections. Members of the human Toll-like receptor family (TLR) are located either on the cell surface or in intracellular vesicles called endosomes. The TLRs are expressed in several immune cells and are also expressed in other cell types, such as fibroblasts, keratinocytes, and endothelial cells. The TLRs, upon activation, dimerize and their intracellular domains recruit proteins of the MyD88 family. The cell surface receptors TLR1, TLR2, TLR4, TLR5, and TLR6 recognize the microbial pathogen-associated molecular pattern through their extracellular domain. LPS mediates the inflammatory response by activating the cell surface Toll-like receptor 4 (TLR4) which then recruits adaptor proteins MyD88, TIRAP, TRIF, and TRAM through its intracellular domain. This activates the intracellular signaling cascade which ultimately results in the expression of inflammatory cytokines. The endosomal receptors TLR3, TLR7, TLR8, and TLR9 recognize the danger-associated molecular pattern and are activated by nucleic acids, such as double-stranded or single-stranded RNA and DNA which may be released from dead cells in damaged tissues. TLR7, TLR8, and TLR9 recruit MyD88 to activate transcription factor IRF3/7 to promote the production of type I interferons. In this study, the inflammation pathway genes comprising the TLR family and chemokine (C-C motif and C-X-C motif) ligands and receptors, and interleukins that were affected by Exo in a chronic low-grade inflammation environment were evaluated. The results indicated that TLR7 expression was regulated in a GAS5-dependent manner. This indicates that release of the noncoding RNA content of hASC exosomes, and particularly lncRNA GAS5, regulated the expression of TLR7. TLR7 expressed on endosomes mediates the expression of type 1 interferons. The results demonstrated that depletion of endogenous GAS5 significantly increased TLR7 expression and the downstream interferon and interleukin targets of TLR7. The molecular mechanism by which GAS5 can promote the expression genes is either via binding to the promoter, as shown for insulin receptor [6], or sequestering proteins, such as glucocorticoid receptors, that promote expression of target genes [23], or via sequestration of miR, which represses expression of target genes [30, 31]. The exact mechanism underlying regulation of expression of TLR7 by GAS5 is being evaluated. Unpublished scratch assay results also showed that depleting endogenous GAS5 in HDF hindered the closure of wound gaps. In this study, the focus was on elucidating the pivotal role of GAS5 carried as a cargo within the exosomes and demonstrated that GAS5 was critical in promoting wound healing in a chronic inflammation environment.

It is estimated that the cost of all wound types in the US is about $31.7 billion [32]. Exosomes derived from human adipose stem cells mediate the recovery and regeneration of wounds. The exosomes are applied topically to the wound and no transfections or carriers are required. Importantly, the results demonstrate that hASC exosomes are a viable therapeutic that accelerates healing of chronic recalcitrant wounds.

### 5. Conclusions

In summary, the results demonstrate that exosomes harvested from human adipose stem cells accelerate the healing of chronic recalcitrant dermal wounds. The lncRNA GAS5 contained in the exosomes is crucial and necessary for the therapeutic potential of hASC exosomes in treating wounds, and, further, GAS5 drives the healing of wounds in an inflammation microenvironment often seen in chronic diseases, such as diabetes and obesity. The results presented here demonstrate that hASC exosomes are a viable therapeutic that accelerates healing of chronic recalcitrant wounds.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Patel, R. S., et al., Adipose-derived stem cells from lean and obese humans show depot specific differences in their stem cell markers, exosome contents and senescence: role of protein kinase C delta (PKCdelta) in adipose stem cell niche. Stem Cell Investig, 2016. 3: p. 2.
2. Ajuwon, K. M. and M. E. Spurlock, Adiponectin inhibits LPS-induced NF-kappaB activation and IL-6 production and increases PPARgamma2 expression in adipocytes. Am J Physiol Regul Integr Comp Physiol, 2005. 288(5): p. R1220-5.
3. Kobori, M., et al., Wedelolactone suppresses LPS-induced caspase-11 expression by directly inhibiting the IKK complex. Cell Death Differ, 2004. 11(1): p. 123-30.
4. Myers, C. L., et al., Induction of ICAM-1 by TNF-alpha, IL-1 beta, and LPS in human endothelial cells after downregulation of PKC. Am J Physiol, 1992. 263(4 Pt 1): p. C767-72.
El Bassit, G., et al., MALAT1 in Human Adipose Stem Cells Modulates Survival and Alternative Splicing of PKCdeltaII in HT22 Cells. Endocrinology, 2017. 158(1): p. 183-195.
6. Shi, Y., et al., Stabilization of lncRNA GAS5 by a Small Molecule and Its Implications in Diabetic Adipocytes. Cell Chem Biol, 2019.
7. Patel, N. A., et al., Long noncoding RNA MALAT1 in exosomes drives regenerative function and modulates inflammation-linked networks following traumatic brain injury. J Neuroinflammation, 2018. 15(1): p. 204.
8. Carter, G., et al., Dysregulated Alternative Splicing Pattern of PKC during Differentiation of Human Preadipocytes Represents Distinct Differences between Lean and Obese Adipocytes. ISRN Obesity, 2013. 2013: p. 9.
9. Dranka, B. P., et al., Assessing bioenergetic function in response to oxidative stress by metabolic profiling. Free Radic Biol Med, 2011. 51(9): p. 1621-35.
10. Cooper, D. R., et al., Human Adipose-Derived Stem Cell Conditioned Media and Exosomes Containing MALAT1 Promote Human Dermal Fibroblast Migration and Ischemic Wound Healing. Adv Wound Care (New Rochelle), 2018. 7(9): p. 299-308.
11. Moss, L. D., et al., Intranasal delivery of exosomes from human adipose derived stem cells at forty-eight hours post injury reduces motor and cognitive impairments following traumatic brain injury. Neurochem Int, 2021. 150: p. 105173.
12. Liu, K., et al., Adipose stem cell-derived exosomes in combination with hyaluronic acid accelerate wound healing through enhancing re-epithelialization and vascularization. Br J Dermatol, 2019. 181(4): p. 854-856.
13. Ma, T., et al., Adipose mesenchymal stem cell-derived exosomes promote cell proliferation, migration, and inhibit cell apoptosis via Wnt/beta-catenin signaling in cutaneous wound healing. J Cell Biochem, 2019. 120(6): p. 10847-10854.
14. An, Y., et al., Exosomes from adipose-derived stem cells and application to skin wound healing. Cell Prolif, 2021. 54(3): p. e12993.
15. Li, X., et al., Exosome Derived From Human Umbilical Cord Mesenchymal Stem Cell Mediates MiR-181c Attenuating Burn-induced Excessive Inflammation. EBioMedicine, 2016. 8: p. 72-82.
16. Lu, Y., et al., Extracellular vesicle-enclosed miR-486-5p mediates wound healing with adipose-derived stem cells by promoting angiogenesis. J Cell Mol Med, 2020. 24(17): p. 9590-9604.
17. Lv, Q., et al., Engineered Human Adipose Stem-Cell-Derived Exosomes Loaded with miR-21-5p to Promote Diabetic Cutaneous Wound Healing. Mol Pharm, 2020. 17(5): p. 1723-1733.
18. He, Z. Y., et al., Long noncoding RNA GAS5 accelerates diabetic wound healing and promotes lymphangiogenesis via miR-217/Prox1 axis. Mol Cell Endocrinol, 2021. 532: p. 111283.
19. Carter, G., et al., Circulating long noncoding RNA GAS5 levels are correlated to prevalence of type 2 diabetes mellitus. BBA Clin, 2015. 4: p. 102-7.
20. Yu, T., et al., Topical insulin accelerates cutaneous wound healing in insulin-resistant diabetic rats. Am J Transl Res, 2017. 9(10): p. 4682-4693.
21. Ribeiro, M. C., et al., Wound healing treatment using insulin within polymeric nanoparticles in the diabetes animal model. Eur J Pharm Sci, 2020. 150: p. 105330.
22. Wang, C., J. Wang, and J. Feng, Local application of low-dose insulin in improving wound healing after deep burn surgery. Exp Ther Med, 2016. 12(4): p. 2527-2530.
23. Kino, T., et al., Noncoding RNA gas5 is a growth arrest- and starvation-associated repressor of the glucocorticoid receptor. Sci Signal, 2010. 3(107): p. ra8.
24. Pichlsberger, M., et al., Systematic Review of the Application of Perinatal Derivatives in Animal Models on Cutaneous Wound Healing. Front Bioeng Biotechnol, 2021. 9: p. 742858.
25. Almadani, Y. H., et al., Wound Healing: A Comprehensive Review. Semin Plast Surg, 2021. 35(3): p. 141-144.
26. Furman, D., et al., Chronic inflammation in the etiology of disease across the life span. Nat Med, 2019. 25(12): p. 1822-1832.
27. Ekuni, D., et al., Lipopolysaccharide-induced epithelial monoamine oxidase mediates alveolar bone loss in a rat chronic wound model. Am J Pathol, 2009. 175(4): p. 1398-409.
28. Wang, D., et al., Low dose of lipopolysaccharide pretreatment can alleviate the in?ammatory response in wound infection mouse model. Chin J Traumatol, 2016. 19(4): p. 193-8.
29. Favere, K., et al., A systematic literature review on the effects of exercise on human Toll-like receptor expression. Exerc Immunol Rev, 2021. 27: p. 84-124.
30. Tan, L., et al., LncRNA GAS5 as miR-26a-5p Sponge Regulates the PTEN/PI3K/Akt Axis and Affects Extracellular Matrix Synthesis in Degenerative Nucleus Pulposus Cells in vitro. Front Neurol, 2021. 12: p. 653341.
31. Zhang, J., et al., LncRNA GAS5 inhibits the proliferation and invasion of ovarian clear cell carcinoma via the miR-31-5p/ARID1A axis. Kaohsiung J Med Sci, 2021. 37(11): p. 940-950.
32. Nussbaum, S. R., et al., An Economic Evaluation of the Impact, Cost, and Medicare Policy Implications of Chronic Nonhealing Wounds. Value Health, 2018. 21(1): p. 27-32.

1. A method of promoting wound healing comprising: administering a composition comprising one or more adipose stem cell derived exosomes to a wound of a subject.

2. A method of treating a wound comprising: administering a composition comprising one or more adipose stem cell derived exosomes to a wound of a subject.

3. (canceled)

4. (canceled)

5. (canceled)

6. The method of claim 1, wherein the wound is a dermal wound, chronic recalcitrant wound cut, laceration or incision.

7. (canceled)

8. (canceled)

9. The method of claim 1, wherein the one or more adipose stem cell derived exosomes is a human adipose stem cell (hASC) exosome.

10. The method of claim 1, wherein the one or more adipose stem cell derived exosomes comprises one or more long noncoding RNAs (lncRNA)

11. The method of claim 10, wherein the one or more lncRNA is growth-arrest specific-5, GAS5, or metastasis-associated lung adenocarcinoma transcript 1 (MALAT1).

12. (canceled)

13. The method of claim 1, wherein the subject has an underlying dermal inflammation.

14. The method of claim 13, wherein underlying dermal inflammation results in impaired natural wound healing.

15. The method of claim 1, wherein the adipose stem cell derived exosome is cell free.

16. The method of claim 1, wherein dermal fibroblasts in the wound take up the one or more adipose stem cell derived exosomes.

17. The method of claim 16, wherein the adipose stem cell derived exosomes are distributed within the cytoplasm and nucleus of the dermal fibroblasts.

18. The method of claim 1, wherein the wound has an accelerated wound closure.

19. The method of claim 1, wherein IL1β and IL6 levels are decreased compared to before administering the composition.

20. The method of claim 1, wherein TLR7, CCL17 and ITGB2 levels are decreased compared to before administering the composition.

21. (canceled)

22. The method of claim 1, wherein the administration is a topical administration.

23. The method of claim 1, wherein the one or more adipose stem cell derived exosomes express CD9, CD63, and CD81.

24. The method of claim 1, wherein the one or more adipose stem cell derived exosomes comprise a targeting moiety expressed on the surface of the one or more adipose stem cell derived exosomes.

25. The method of claim 24, wherein the targeting moiety comprises a peptide which binds to a moiety present on the cell to be targeted.

26.-39. (canceled)

40. A subcutaneous-derived adipose stem cell derived exosome comprising lncRNA growth-arrest specific-5 (GAS5) and/or metastasis-associated lung adenocarcinoma transcript 1 (MALAT1), wherein GAS5 and/or MALAT1 are overexpressed.

41. The subcutaneous-derived adipose stem cell derived exosome of claim 40, wherein the subcutaneous-derived adipose stem cell derived exosome comprises CD9, CD63, and CD81 on its surface.

42.-50. (canceled)
